(19) United States
(12) Patent Application Publication
(10) Pub. No.: US 2024/0268827 A1
(43) Pub. Date: Aug. 15, 2024

(54) APPLICATOR TOOL CAPABLE OF USE WITH FORCE MODULATING TISSUE BRIDGE, AND ASSOCIATED SYSTEMS, METHODS AND KITS

(71) Applicant: BRIJ Medical, Inc., Marietta, GA (US)

(72) Inventors: Felmont F. EAVES, Atlanta, GA (US); Gary W. Knight, Lebanon, OH (US); Jacob E. Hood, Marietta, GA (US)

(21) Appl. No.: 18/644,717

(22) Filed: Apr. 24, 2024

Related U.S. Application Data
(63) Continuation of application No. PCT/US2022/048359, filed on Oct. 31, 2022.
(60) Provisional application No. 63/274,132, filed on Nov. 1, 2021.

(51) Int. Cl. A61B 17/08

(52) U.S. Cl. CPC A61B 17/083; A61B 17/085

(57) ABSTRACT

Catch parts of a tool include first and second shanks, and first and second outer protrusions respectively extending outwardly from the shanks. For streamlining disengaging of the tool from a medical article, the tool includes at least one inner protrusion extending inwardly from a respective shank. The inner protrusion can engage at least one portion of the medical article to streamline removal of the tool from the medical article.

### CROSS-REFERENCE TO RELATED APPLICATIONS

This application is a continuation of International Application No. PCT/US2022/048359, filed Oct. 31, 2022, which claims the benefit of U.S. Provisional Patent Application No. 63/274,132, filed Nov. 1, 2021. Each of International Application No. PCT/US2022/048359, filed Oct. 31, 2022, and U.S. Provisional Patent Application No. 63/274,132, filed Nov. 1, 2021, is incorporated herein by reference in its entirety.

### BACKGROUND

Applicator tools capable of use with force modulating tissue bridges are known, for example, from United States Patent Application Publication No. 2019/0133582.

It is believed to be possible in some circumstances for an inexperienced user of one type of an applicator tool to accidently have trouble disengaging the applicator tool from a force modulating tissue bridge that has been applied to tissue using the applicator tool. Accordingly and as an example, there may be a desire for an applicator tool configured in a manner that seeks to streamline removing the applicator tool from a force modulating tissue bridge.

### SUMMARY

An aspect of this disclosure is the provision of a tool configured to releasably engage and interact with a medical article. As an example, the tool can be an applicator tool, the medical article can be a force modulating tissue bridge, and the tool can be configured in a manner that seeks to streamline removing the applicator tool from the force modulating tissue bridge that has been applied to tissue using the applicator tool.

As an example, the tool can include first and second levers pivotably connected to one another, and first and second catch parts configured to at least partially releasably connect the tool to the medical article. The first and second catch parts can respectively include first and second shanks respectively connected to the first and second levers, and first and second outer protrusions (e.g., latches or latch parts) respectively extending outwardly from the first and second shanks. The tool can be configured to be connected to the medical article at least partially in response an increased distance between the first and second outer protrusions. The tool can be configured to be removed from the medical article at least partially in response to a reduced distance between the first and second outer protrusions. For streamlining removal of the tool from the medical article, the tool can further include at least one backslope and/or inner protrusion (e.g., blocking mechanism or deflection restrictor) extending inwardly from one of the first and second shanks. For example, the tool can include first and second inner protrusions (e.g., blocking mechanisms or deflection restrictors) respectively extending inwardly from the first and second shanks.

The one or more inner protrusions, for example the first and second inner protrusions, can be configured to engage (e.g., slidingly engage) respective portions (e.g., edges of holes) of the medical article to restrict relative movement between and/or how close the first and second catch parts can be to one another while the tool is being removed from the medical article. As a more specific example, the first and second inner protrusions can be configured to slidingly engage the respective portions of the medical article to restrict the tool from inadvertently being in a mechanically interfering arrangement with (e.g., reconnecting with) the medical article by way of any occurrence of the first and second catch parts becoming too close to one another. As a further specific example, the first and second inner protrusions can be configured to slidingly engage the respective portions of the medical article to restrict the catch parts from engaging beneath respective portions of the medical article while the tool is being removed from the medical article.

The first and second inner protrusions can include engagement surfaces configured to slidingly engage the respective portions of the medical article while the tool is being removed from the medical article. The engagement surfaces can extend vertically or be inclined (e.g., extend outwardly in a downward direction). The engagement surfaces can be planar or in any other suitable configurations. The medical article can include holes into which the shanks extend, and the engagement surfaces can slidingly engage respective edges of the holes.

Opposite sides of the tool can be symmetrical or unsymmetrical with respect to one another. For example and as alluded to above, one of the first and second inner protrusions can be omitted.

The foregoing summary provides a few brief examples and is not exhaustive, and the present invention is not limited to the foregoing examples. Various other features, aspects, and advantages of the present invention will be evident from the following description and accompanying drawings.

### BRIEF DESCRIPTION OF THE DRAWINGS

The drawings are provided as examples and may not be drawn to scale. The present invention may be embodied in many different forms and should not be construed as limited to the examples depicted in the drawings.

FIG. 1 is a top perspective view of an exemplary medical article (e.g., force modulating tissue bridge) for treating a wound and/or scar tissue, in accordance with an embodiment of this disclosure.

FIG. 2 is a top plan view of the tissue bridge of FIG. 1.

FIG. 3 is a side elevation view of the tissue bridge of FIG. 1.

FIG. 4 is a cross-sectional view taken along line 4-4 of FIG. 2.

FIG. 5 is an end elevation view of the tissue bridge of FIG. 1.

FIG. 6 is an exploded top perspective view of selected exemplary layers of the tissue bridge of FIG. 1.

FIG. 7 is a perspective view of an exemplary tool configured for being used to releasably engage, manipulate, and/or apply the tissue bridge of FIG. 1, wherein a perspective view of the tool from the opposite side depicted in FIG. 7 can be a mirror image of FIG. 7, in accordance with a first embodiment of this disclosure.

FIG. 8 is a front elevation view of the tool of FIG. 7, and a rear elevation view can be identical.

FIG. 9 is a right-side elevation view of the tool of FIG. 7, and a left-side elevation view can be identical.

FIG. 10 is a relatively enlarged perspective view of a lower portion of the tool of FIG. 7, wherein a perspective view of the lower portion of the tool from the opposite side depicted in FIG. 10 can be a mirror image of FIG. 10.

FIG. 11 depicts an example of the tool of FIG. 7 mounted to (e.g., releasably engaged with) the tissue bridge of FIG. 1, wherein a perspective view of the system of FIG. 11 from the opposite side depicted in FIG. 11 can be a mirror image of FIG. 11, in accordance with an embodiment of this disclosure.

FIGS. 12A through 12F depict a sequence of steps of an exemplary method of using the tool of FIG. 7 to remove the tissue bridge of FIG. 1 from a tray, in accordance with an embodiment of this disclosure.

FIGS. 12G through 12M depict a sequence of steps of an exemplary method of using the tool of FIG. 7 to apply the tissue bridge of FIG. 1 to a wound, and then releasing the tool from its engagement with the tissue bridge, in accordance with an embodiment of this disclosure.

FIG. 13 is a front elevation view of the tool of FIG. 7 that further schematically includes reference lines that serve as a basis for at least partially describing features of the tool.

FIG. 14 is a front elevation view of a tool of a second embodiment of this disclosure, wherein FIG. 14 further schematically includes reference lines that serve as a basis for at least partially describing features of the tool.

FIG. 15 depicts an example of the tool of FIG. 14 in combination with the tissue bridge of FIG. 1 or a suitable variant thereof, wherein the tissue bridge is cross sectioned substantially similarly to in FIG. 4, and FIG. 15 further schematically includes reference lines that serve as a basis for at least partially describing features of the tool.

FIG. 16 is a partial view (i.e., a relatively enlarged view of a portion of FIG. 15) schematically depicting angles between areas of sliding contact occurring between respectively engaged portions of the tool and tissue bridge in accordance with the second embodiment.

FIG. 17 depicts a lower portion of a tool, and schematically depicts aspects the tool being removed from a tissue bridge, in accordance with a third embodiment of this disclosure.

### DETAILED DESCRIPTION

Embodiments are described below and illustrated in the accompanying figures, in which like numerals refer to like parts throughout the several views. The embodiments described provide examples and should not be interpreted as limiting the scope of the invention. Other embodiments, and modifications and improvements of the described embodiments, will occur to those skilled in the art and all such other embodiments, modifications, and improvements are within the scope of the invention. As another example of the breadth of this disclosure, it is within the scope of this disclosure for one or more of the terms “substantially,” “about,” “approximately,” and/or the like, to qualify each of the adjectives and adverbs of the Detailed Description section of this disclosure, as discussed further below.

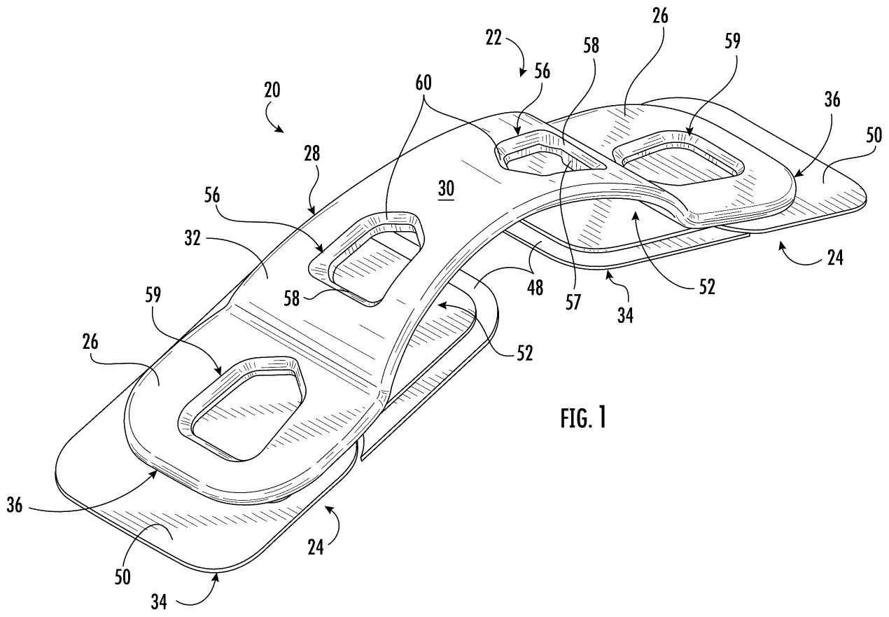

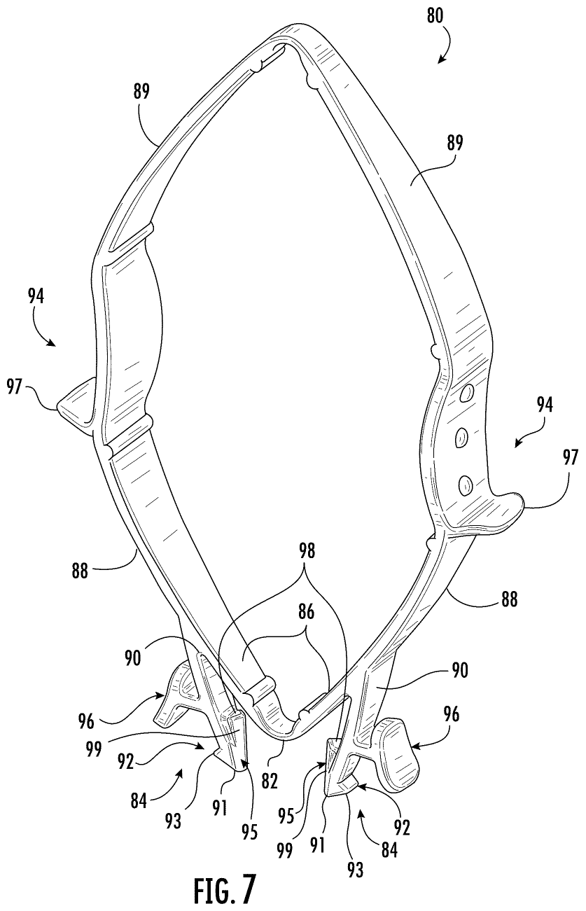

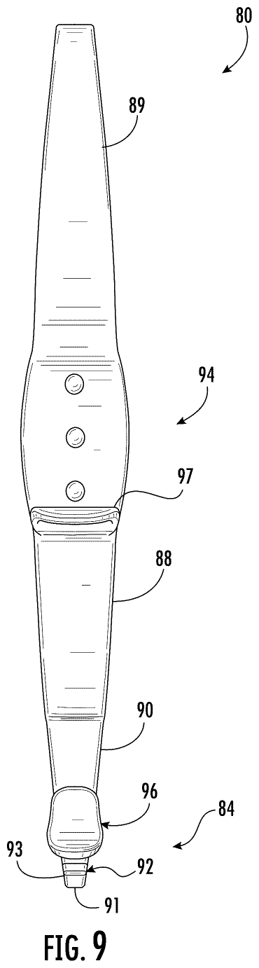

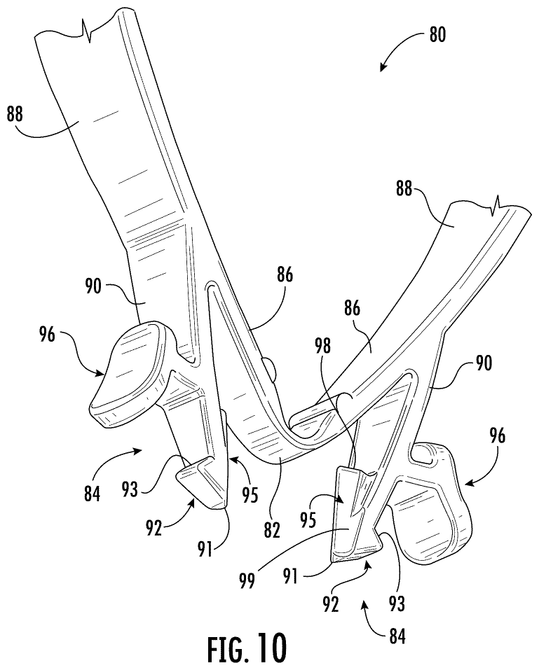

As an example that is discussed in greater detail below, an applicator tool 80 (FIGS. 7-10 and 13-14) can be used to apply a medical article 20 (FIGS. 1-6) to a patient's tissue, and the applicator tool and medical article can be cooperatively configured to streamline removal of the applicator tool from the medical article. See, e.g., the applicator tool's one or more backslopes and/or inner protrusions 95 (FIGS. 7-8, 10, and 13-14), which may be referred to as deflection restrictors 95 for ease of understanding in this Detailed Description section of this disclosure, as discussed further below.

FIGS. 1-5 depict an at least partially elastic (e.g., generally elastic) medical article 20 in its undeformed or at-rest configuration (e.g., relaxed state), in accordance with an exemplary embodiment. The medical article 20 may optionally be referred to as a force modulating tissue bridge 20, or simply tissue bridge 20, and the tissue bridge may be more generally referred to as a medical article. In the following, an example of a method of using the tissue bridge 20 is briefly described, and thereafter the tissue bridge, applicator tool, and other aspects of this disclosure are described in greater detail.

The tissue bridge 20 can be mounted to tissue such as, but not limited to, a surface of a patient's skin, for example, the outer surface of the patient's epidermis. The tissue bridge 20 is typically mounted so that it extends across and at least partially covers a wound and/or scar of a patient. The tissue bridge 20 may comprise elastic material, and prior to the tissue bridge being mounted on the patient, the tissue bridge can be generally elastically deformed from its undeformed or at-rest configuration to a strained, deformed, at least partially flattened, and/or extended configuration. The tissue bridge 20 can at least begin to be mounted to the tissue (e.g., skin tissue of a patient) such that a central section of the tissue bridge extends across a wound and/or scar while the tissue bridge is maintained in its extended configuration. After being at least partially mounted in its extended configuration, the tissue bridge 20 can be allowed to generally elastically reconfigure from its extended configuration, at least partially, toward its at-rest configuration, which may, for example, reduce tension in the tissue, help close the wound, help inhibit wound reopening, and/or inhibit scar disfiguring (e.g., widening), as will be discussed in greater detail below. The generally elastic material of the tissue bridge 20 may be configured such that the tissue bridge is biased toward its at-rest configuration (e.g., relaxed state).

The tissue bridge 20 may comprise a generally elastic body 22 (e.g., at least partially elastic) and one or more multi-layer foot pads 24 mounted to the body, although in some examples one or more of the foot pads and/or portions thereof can be omitted (e.g., a footpad may consist of, or consist essentially of, a single layer). The body 22 can be generally referred to as and/or generally function as a backbone or other suitable structure configured to movably connect two or more of the foot pads 24 to one another.

The body 22 may include at least two flanges 26 (e.g., feet) respectively extending obliquely, for example outwardly and downwardly, from opposite lower portions of a central section or arch 28 of the body. Each of the flanges 26 can be planar, or they can be substantially or about planar since it may not be critical that the flanges be exactly planar. The flanges 26 can extend divergently relative to one another, and obliquely relative to one another. The arch 28 can include a central spanning section 30, and lower sections 32 respectively extending downwardly from opposite portions of the spanning section. The lower sections 32 of the arch 28 can optionally be configured as and/or referred to as shoulders 32. The flanges 26 can respectively extend obliquely, for example outwardly and downwardly, from lower portions of the shoulders 32. The shoulders 32 can provide a smoothly curved transition between the spanning section 30 of the arch 28 and the flanges 26. In the embodiment shown in FIGS. 1-6, the spanning section 30 of the arch 28 has a relatively low profile and is at least generally arcuate, which can be advantageous for an active person having the tissue bridge 20 mounted on their skin.

Each of the parts of the tissue bridge 20 will typically be constructed of suitable medical-grade materials. For example, the body 22 can be an injection-molded or mechanically thermoformed, unitary (e.g., single-piece) article such that the spanning section 30, shoulders 32, and flanges 26 can be formed together as a single article from an injection-moldable or formable, generally elastic material such as, but not limited to, polycarbonate, or any other suitable injection-moldable or formable material.

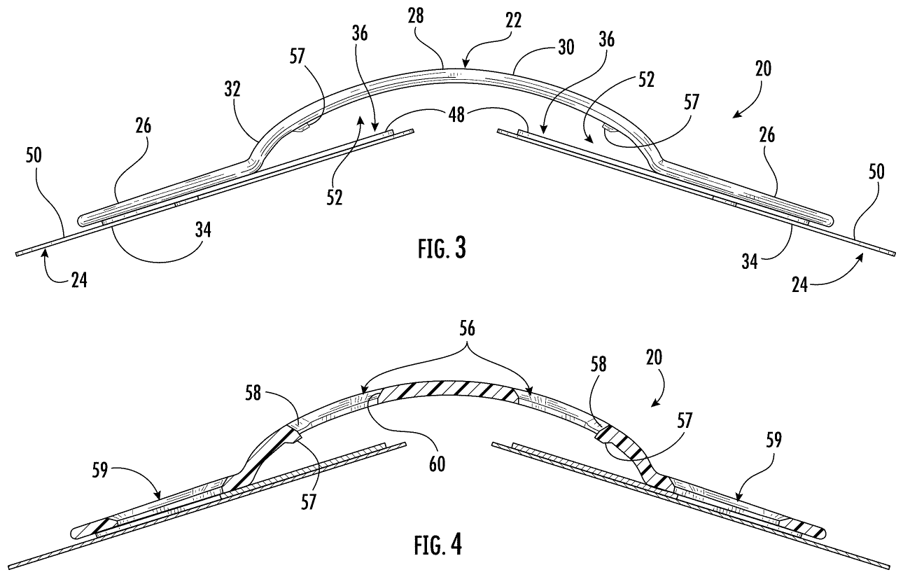

Referring to FIG. 3, each of the spanning section 30, shoulders 32, and flanges 26 can be about the same thickness, or alternatively the thickness of the body 22 can vary along its length.

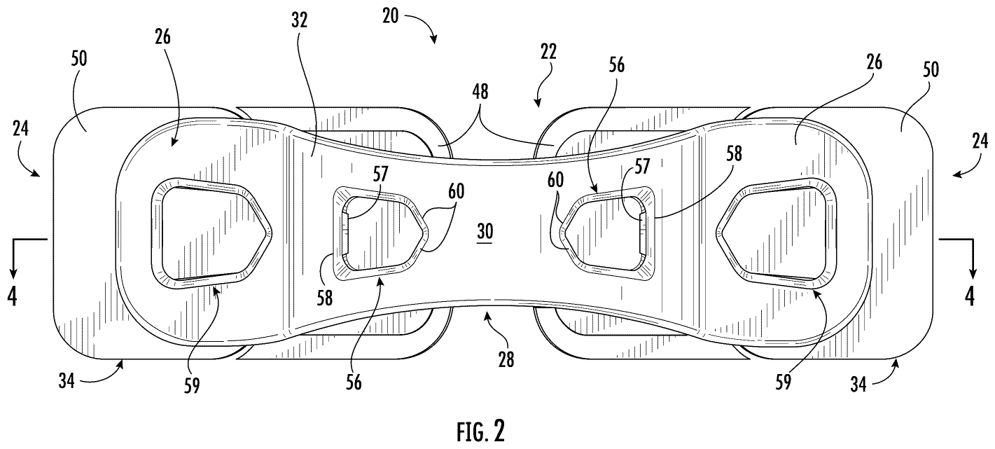

Referring to FIG. 2, the width of the body 22 can, for example, taper along its length, so that the spanning section 30 is relatively narrow (e.g., has a narrowed waist) as compared to the shoulder 32 and flanges 26, so that the spanning section can be more readily deformed as compared to the shoulders and flanges. For example, the side edges of the spanning section 30 can be inwardly curved or concave, as shown in FIG. 2, or they may have a stepped or other suitable configurations. Alternatively, the side edges of the spanning section 30 can extend generally or substantially straight in a top plan view of the tissue bridge 20, or they can extend in any other suitable manner.

As shown in FIGS. 1-3, the foot pads 24 can be spaced apart from one another, and the foot pads can be fixedly mounted to the flanges 26. Each foot pad 24 can be or include a mat, laminate, or other suitable structure comprising one or more layers of material. For example, referring to FIG. 3, each foot pad 24 includes an outer layer or sheet 34 configured to be attached to tissue (e.g., skin tissue), and an inner layer or sheet 36 positioned between, and fixedly connected to each of, the outer sheet 34 and the respective flange 26.

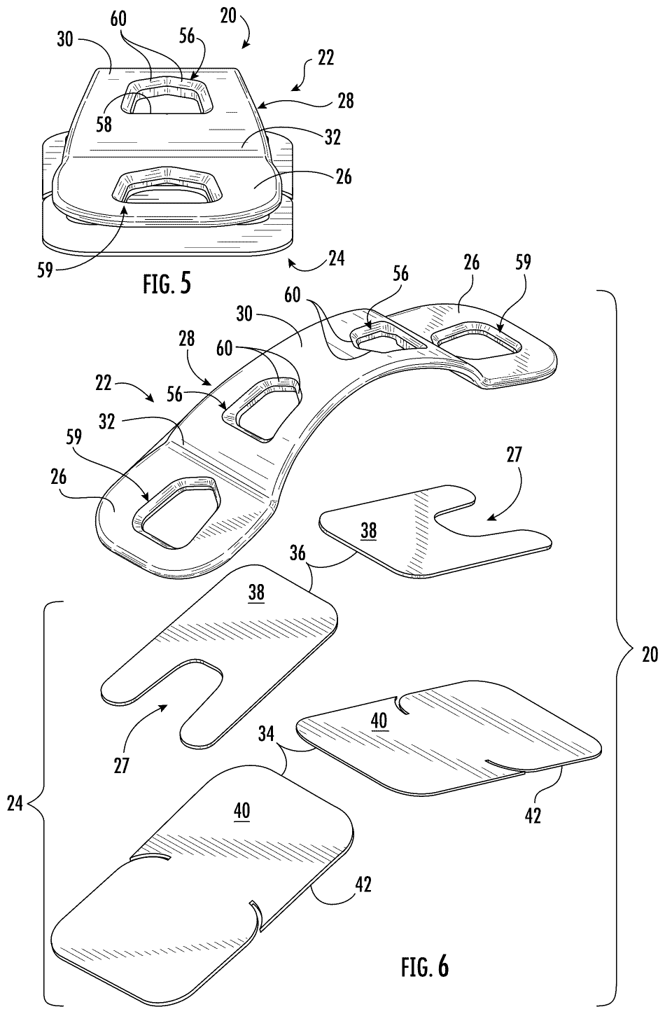

Referring to the exploded view of FIG. 6, the tissue bridge 20 can include respective inner, intermediate, and outer adhesive layers 38, 40, 42. The inner adhesive layers 38 can be between and fixedly connect the inner sheets 36 to the flanges 26, the intermediate adhesive layers 40 can be between and fixedly connect the outer sheets 34 to the inner sheets, and the outer adhesive layers 42 can be on the outer sides of the outer sheets for attaching the tissue bridge 20 to tissue (e.g., a patient's skin), as will be discussed in greater detail below.

The outer and inner sheets 34, 36 can be provided, for example, by die cutting from appropriate webs or larger sheets of material, such as fabric or cast microporous polymeric sheet for the outer sheets 34, and an extruded polymer or plastic sheet for the inner sheets 36. The outer sheets 34 can be made of suitable fabric materials, cast materials, films, or other materials of the type from which skin-contact layers of bandages or other wound dressings are formed, or any other suitable material. The plastic inner sheets 36 can be made of suitable materials such as, for example, polyethylene, polyethylene terephthalate, or any other suitable materials. The inner and intermediate adhesive layers 38, 40 can respectively comprise adhesive materials that are compatible with the materials being connected thereby. The outer adhesive layer 42 (e.g., patient contact adhesive) can be, for example, adhesive material of the type that is typically used as an adhesive backing for bandages or other wound dressings. In an exemplary embodiment, the outer adhesive layer 42 can have a lower adhesive strength than the inner and intermediate adhesive layers 38, 40, such as when the tissue bridge 20 is to be removably mounted to tissue (e.g., a patient's skin).

The body 22 and the inner sheet 36 may have a higher modulus of elasticity (e.g., formed from stiffer material) than the outer sheet 34. More generally, the body 22 and the inner sheet 36 can be stiffer than the outer sheet 34 due to a number of factors, such as being larger, thicker, comprising material having a higher modulus of elasticity, and/or being constructed to have an apparent modulus of elasticity. Similarly, the body 22 can have a higher modulus of elasticity than the foot pads 24.

Referring to FIG. 3, inner extensions 48 of the outer and inner sheets 34, 36 can extend into the central area over which the arch 28 extends such that the inner extensions 48 are neither superposed by nor coextensive with the flanges 26. More generally, each foot pad 24 can include at least one extension 48 that extends into the central area over which the arch 28 extends. The inner extensions 48 may be referred to as medial extensions 48, for example, since they extend toward the middle of the area over which the arch 28 extends. The inner or medial extensions 48 can be configured so that they at least partially resist longitudinal compression when the tissue bridge 20, in its extended configuration, is mounted to tissue (e.g., skin tissue), and to generally elastically reconfigure from its extended configuration at least partially toward its at-rest configuration. Accordingly, the inner or medial extensions 48 can be referred to as medial struts 48. A gap can be defined between adjacent ends of the medial extensions 48, and the gap can be configured, for example, so that the adjacent ends of the medial extensions 48 are spaced apart from one another and do not contact one another.

Referring to FIGS. 1-3, for each foot pad 24, one or more (e.g., four) margins or outer extensions 50 of the outer sheets 34 can extend outwardly beyond the inner sheet 36 such that the margins or outer extensions are neither superposed by nor coextensive with the inner sheet 36. Each medial strut 48 may include inner extensions of both sheets 34, 36, but one or more layers or sheets of the medial strut 48 can be omitted, such that each medial strut can be formed of one layer of material.

As shown in FIGS. 1-3, the medial struts 48 can be spaced apart from (e.g., at least partially spaced apart from) the arch 28 and extend into the central area over which the arch extends, so that gaps or receptacles 52 are at least partially defined between the medial struts and the arch. The receptacles 52 can at least partially define, or be at least part of, catch parts configured for interacting with corresponding features of an applicator tool that may be used, for example, in the mounting of the tissue bridge 20 to tissue (e.g., a patient's skin). For example, the tissue bridge 20 can include one or more catch parts, and the catch parts can respectively comprise the receptacles 52. A variety of differently configured catch parts are within the scope of this disclosure. Exemplary applicator tools and related features are discussed in greater detail below.

As shown in FIGS. 1-5, the body 22 can include at least two catch parts that further comprise inner holes 56 that extend through the body 22 and are open to the receptacles 52. FIG. 4 is a cross-sectional view of the tissue bridge taken along line 4-4 of FIG. 2. The inner holes 56 can be defined in the arch 28, or more specifically the inner holes 56 can be positioned in opposite end portions of the spanning section 30. The inner holes 56 can be open to the central area over which the arch 28 extends or more specifically the inner holes can be open to the receptacles 52, and the medial struts 48 can extend beneath the inner holes. The inner holes 56 can have any suitable shape or configuration. For example, the inner holes 56 can be generally polygonal, or generally rectangular (e.g., rectangular extending to a chevron-shape), with an outer edge 58 that defines the inner hole 56 extending crosswise to the length of the arch.

The catch parts can further include outer edges 58 (see, e.g., FIGS. 1-5) and protrusions or ribs 57 (e.g., keepers) extending downwardly from (e.g., downwardly from proximate) the outer edges 58. Each respective outer edge 58 and rib 57 can extend parallel, or more generally substantially parallel or about parallel, to the boundary between the spanning section 30 and the respective shoulder 32. In other words, the outer edge 58 and rib 57 can extend perpendicular to, or more generally substantially perpendicular to or about perpendicular to, the lengthwise or longitudinal axis of the body 22. In addition, the inner holes 56 can reduce the area or volume of the outer portions of the spanning section 30 in a manner that enhances the deformability of the outer portions of the spanning section.

Each of the inner holes 56 of the tissue bridge body 22 can include one or more inner edges 60 opposite from its outer edge 58. More specifically, each tissue bridge inner hole 56 can be partially defined by a pair of inner edges 60 that extend convergently toward a junction or vertex to define a corner. This corner defined at or by the convergence of the inner edges 60 may be referred to as a concave corner for ease of understanding. The concave corner between the tissue bridge inner edges 60 can be generally chevron-shaped, V-shaped, and/or in any other suitable configuration, as discussed further below. Alternatively, each tissue bridge inner hole 56 may include a single straight inner edge 60 or other suitable inner edge(s).

The body 22 can additionally or alternatively have one or more other holes formed therein or therethrough. For example, outer holes 59 can extend through the foot plates or flanges 26 (see, e.g., FIGS. 1-5). Referring to the exploded view of FIG. 6, the adhesive layers 38, 40 and inner sheets 36 can further include lower holes 27. The lower holes 27 can be open to the outer holes 59, and the lower holes 27 can also extend outwardly to be open at the outer edges of the adhesive layers 38, 40 and inner sheets 36. The outer holes 59 can have any suitable shape. For example, the outer holes 59 can be generally polygonal, or generally rectangular. The lower holes 27 can be shaped complementary to the outer holes 59.

The tissue bridge 20 can, for example, be configured so that each flange 26 has opposite upper and lower surfaces that are each larger than a thickness defined between the upper and lower surfaces of the flange; each foot pad 24 has opposite upper and lower surfaces that are each larger than a thickness defined between the upper and lower surfaces of the foot pad; and for each flange 26 and the respective foot pad 24 connected thereto, the upper surface of the foot pad and the lower surface of the flange can face toward one another, can be superposed with one another, can be parallel (e.g., substantially parallel) to one another, and/or can be directly connected to one another by the inner adhesive layers 38 positioned therebetween.

FIGS. 7-10 depict various views of an exemplary applicator mechanism in the form of an applicator tool 80 that can be used, for example, to manipulate a tissue bridge 20 (FIGS. 1-6) or another suitable medical article, for example, as part of an exemplary system and/or method of mounting the tissue bridge to tissue (e.g., a patient's skin), as discussed further below. The applicator tool 80 together with one or more of the tissue bridges 20 can be supplied together as part of a kit.

For example, the applicator tool 80 can include one or more parts or features that can be spaced apart from one another and can be configured to releasably engage the tissue bridge 20. The one or more parts or features of the applicator tool 80 that are configured to releasably engage the tissue bridge 20 can comprise one or more catch parts 84 (e.g., catch pins). The applicator tool 80 can include a tool feature, structure, and/or surface 82 positioned between the tool catch parts 84. The surface 82 may be used for bearing or engaging against a corresponding portion of a tissue bridge, as discussed further below. The applicator tool 80 can further include a reconfigurable frame connecting the tool surface 82 and tool catch parts 84 to one another.

The frame of the applicator tool 80 can include a reconfigurable linkage (e.g., one or more links 86) connecting the tool surface 82 and tool catch parts 84 to one another. The frame of the applicator tool 80 can further include one or more levers 88 comprising and/or extending upwardly from the links 86. The applicator tool 80 can be configured so that when the surface 82 faces downwardly, the catch parts 84 extend downwardly from the linkage (e.g., link(s) 86), and the levers 88 extend upwardly from the linkage. The links 86 and the levers 88 can be cooperatively configured so that at least portions of the catch parts 84 move away from one another, and the surface 82 moves toward a line extending from one to the other of the catch parts 84, in response to at least portions of the levers 88 being moved toward one another, as will be discussed in greater detail below.

The tool surface 82 can be a lower end face of a pivotable junction (e.g., flexible joint, living hinge, area of reduced thickness, or the like) between the links 86. The tool catch parts 84 can include shanks 90 (e.g., rods, shafts, or other suitable structures) extending from upper end portions of the links 86 and/or lower end portions of the levers 88. The links 86 can be referred to as lower sections of the levers 88.

Each tool catch part 84 can further include at least one outer protrusion 92 (e.g., latch or latch part) or other suitable latching feature(s) extending outwardly from the lower end portion of the shank 90 in a direction generally crosswise to the length of the shank. The outer latching protrusions 92 can face away from one another. Each outer protrusion 92 can be generally rectangular (e.g., a plane shape), include an engagement shoulder or surface 93 extending outwardly from the shank 90, and terminate at or about a lower tip 91. An approximately ninety-degree angle (e.g., within a range of fifteen degrees) can be defined between the engagement surfaces 93 (e.g., shoulders) and the outer sides of shanks 90. The links 86 and the levers 88 can be cooperatively configured so that the outer protrusions 92 move away from one another in response to at least portions of the levers 88 being moved toward one another. As alluded to above, the outer protrusions 92 may at least partially perform a latching function with respect to a tissue bridge 20 (as will be discussed further below), and, thus, they may be referred to as latches 92 for ease of understanding in this Detailed Description section of this disclosure.

As best understood with reference to FIGS. 7-8 and 10, the applicator tool 80 can further includes one or more inner protrusions 95 (e.g., backslopes, deflection-restricting backslopes, ejectors, deflection protrusions, deflection restrictors, etc.) mounted to, or formed with, the shanks 90. As compared to the outer protrusions or latches 92, the inner protrusions 95 extend inwardly from opposite sides of the shanks 90. Notably, inner protrusions 95 extend inwardly from inner sides of the shanks 90 and define backslopes that can engage (e.g., slidingly engage) against the tissue bridge inner edges 60 (FIGS. 1-2 and 4-6) without extending too far under the tissue bridge inner edges or otherwise impinging (e.g., restricting) removal of the tool 80 from the tissue bridge 20, as will be discussed in greater detail below. The inner protrusions 95 may at least partially perform a deflection-restricting function with respect to a releasing or disconnecting of the tool 80 from tissue bridge 20 (as will be discussed further below), and, thus, they may be referred to as deflection restrictors 95 for ease of understanding in this Detailed Description section of this disclosure. Optionally, for each of the catch parts 84 (e.g., catch pins), the corresponding deflection restrictor 95 may be referred to as a part or portion of the catch part, for example the deflection restrictor 95 may be referred to as a backslope of the catch part (e.g., catch pin). One of the deflection restrictors 95 depicted in FIGS. 7-8 and 10 can be omitted, as discussed further below.

With continued reference to FIGS. 7-8 and 10, each inner protrusion or deflection restrictor 95 can include an inner shelf or inner shoulder 98 (e.g., a horizontally extending, generally horizontally extending, or inclined portion that may be flat) extending inwardly from the respective shank 90 at a point that is above (e.g., a higher elevation than) the outer engagement shoulders 93 of the latching protrusions 92. Each of the deflection restrictors 95 can include an at least one upright portion or engagement face 99 (e.g., a upright backslope extending downwardly from the inner shoulder 98). Each backslope or engagement face 99 may or may not be inclined relative to a variety of different frames of reference, as discussed further below with reference to a variety of different angles. Each deflection restrictor 95 can be downwardly tapered and extend to the shank tip 91.

As best understood with reference to FIGS. 7-8 and 10, for each inner protrusions or deflection restrictor 95, its shoulder 98 can be triangular or at least partially triangular, and a pair of upright engagement faces 99 (e.g., backslopes) can extend downwardly from respective edges of the shoulder 98. The engagement faces 99 can extend convergently toward an upright junction or elongate vertex to define an upright corner. This corner defined at or by the convergence of the engagement faces 99 may be referred to as a convex corner for ease of understanding. The convex corner between the engagement faces 99 can be generally chevron-shaped, V-shaped, and/or in any other suitable configuration, as discussed further below. The engagement faces 99 can each be flat or planar, or in any other suitable shape. Alternatively, each deflection restrictor 95 may include a single flat engagement face 99 or other suitable engagement surface(s).

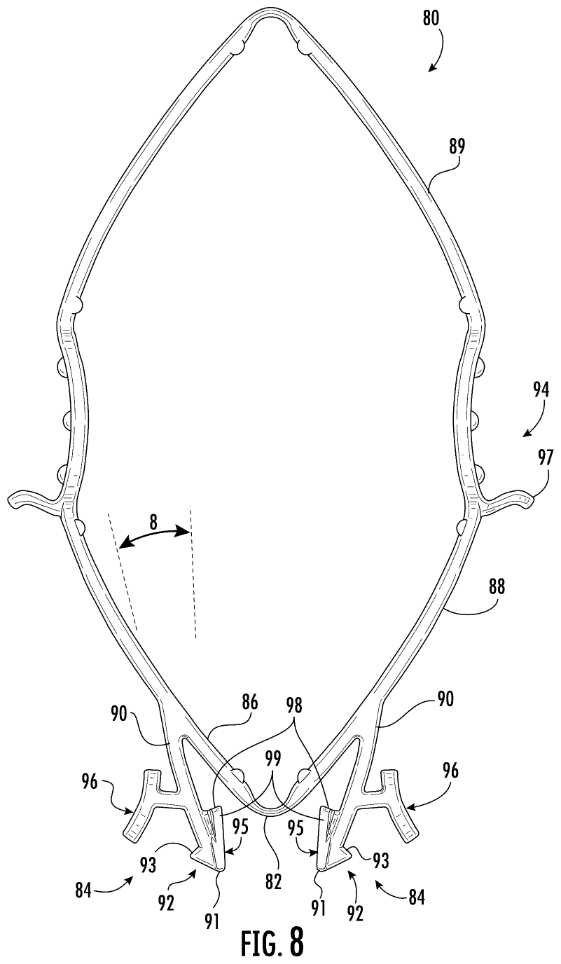

The tool 80 may define one or more angular relationships, one of which is discussed immediately below, and others of which are discussed further below. For example and as schematically depicted in FIG. 8, for each tool catch part 84 and its associated deflection restrictor 95, an angle designated by numeral 8 can be defined therebetween. For example, the angle 8 can be defined between the shank 90 (e.g., a side of the shank, axis of the shank, and/or lengthwise axis of the shank) of the tool catch part 84 and the backslope (e.g., the one or more engagement faces 99) of or defined by the deflection restrictor 95. In one aspect of this disclosure, at least in the Detailed Description section of this disclosure, the engagement faces 99, or the like, may optionally be referred to as backslopes (e.g., for ease of understanding) because in the embodiments depicted in the drawings the angle 8 is a non-zero angle, at least in the at-rest configurations of the tools 80. That said, a variety of different configurations are within the scope of this disclosure. For example, it is believed that there may be a suitably configured tool 80 (not depicted in the drawings) in which the deflection restrictors 95 are present but associated angles 8 may not be present or may be close to 0 degrees (e.g., due to the shanks 90 being configured such that the shank axes, or the like, are parallel to the engagement faces 99, or the like).

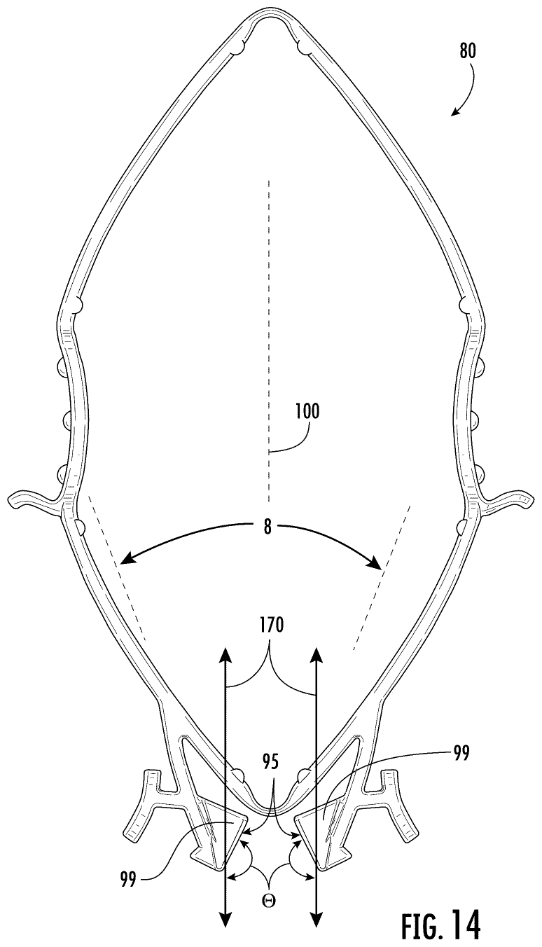

In the example depicted in FIG. 8, it is believed that the angle 8 can be at least about 5 degrees, about 14 degrees, less than about 30 degrees, in a range of from about 5 degrees to about 30 degrees, from about 5 degrees to about 25 degrees, from about 8 degrees to about 20 degrees, from about 10 degrees to about 18 degrees, or any values or subranges therebetween, including both with and without the adjective “about.” More generally and as partially discussed further below with reference to FIG. 14, it is believed the angle 8 (see, e.g., both FIG. 8 and FIG. 14) can be in a range of from about 0 degrees to about 90 degrees, from about 5 degrees to about 70 degrees, from about 10 degrees to about 60 degrees, from about 20 degrees to about 50 degrees, from about 30 degrees to about 40 degrees, or any values or subranges therebetween, including both with and without the adjective “about.”

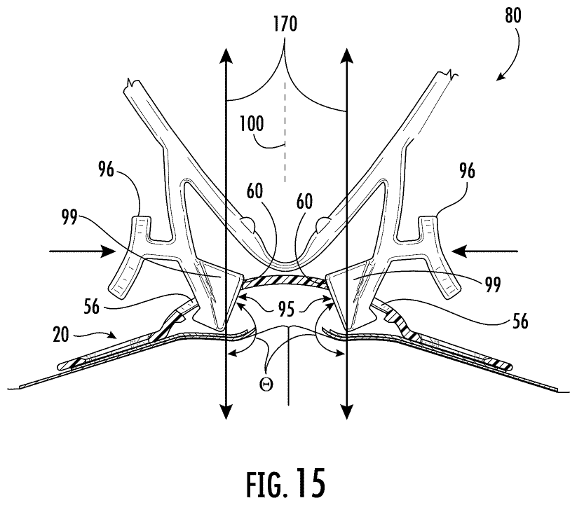

Rather than a generally horizontally extending or inclined flat shelf or shoulder 98, upper portions of the deflection restrictors 95 can alternatively include different shapes or configurations (e.g., angles, curves, fillets, rounds, chamfers, etc.), non-limiting examples of which are shown in FIGS. 14 and 15. Upper portions of the deflection restrictors 95 could, for example, also be configured to interact with corresponding portions of the tissue bridge 20 and/or be configured so as to provide clearance during use. Similarly, the downwardly extending backslope or engagement surfaces or faces 99 of the deflection restrictors 95 may alternatively include different shapes or configurations (e.g., the backslope or engagement surfaces 99 can be straight, can curve inwardly or outwardly, can extend at an angle that tapers inwardly or outwardly from the upper portion or shelf, etc.).

In the embodiment depicted in FIGS. 7-10, opposite release tabs or pads 96 (i.e., finger pads, touch pads, release tabs, etc.) include and/are supported by extensions, shafts, rods and/or other suitable structures extending outwardly from outer sides of shanks 90. As will be discussed in greater detail below, one or more of the release pads 96 and/or other suitable features can be used (e.g., manually manipulated by a user) to cause the catch parts 84 to move closer to one another so that the catch parts 84 can be passed through (e.g., out of) or otherwise released from corresponding holes or corresponding catch parts of the tissue bridge 20. As another example discussed further below, one of the catch parts 84 can be moved closer to the other of the catch parts so that the catch parts can be (e.g., serially can be) passed through (e.g., out of) or otherwise released from holes or corresponding catch parts of the tissue bridge 20. The release pads 96 are optional and may be omitted, or configured differently than depicted in the drawings.

The links 86 can extend obliquely, outwardly, and upwardly from the tool surface 82 respectively to upper portions of the shanks 90. The levers 88 can extend obliquely, outwardly, and upwardly respectively from upper portions of the shanks 90.

The levers 88 can define or comprise handles 94. The handles 94 can, for example, be inwardly recessed, arcuate, and/or concave sections of the levers 88, although differently configured handles are within the scope of this disclosure. As an example, handles 94 each having an outer stop surface 97 extending crosswise to the length of the applicator tool 80 in a manner that seeks to restrict a user's fingers from inadvertently sliding downwardly out of the handle 94. The stop surface 97 can be an upper surface of a post or other suitable protrusion. As another example, the stop surface 97 can be a lower portion of a concave, arcuate or otherwise suitable curved surface. The handles 94 can also include, for example, knurling configured in a manner that seeks to restrict a user's fingers from inadvertently sliding downwardly out of or away from the handles 94. As depicted, for example, the knurling can be in the form of protrusions or hemispherical bumps, although other features can be included for restricting a user's fingers from inadvertently sliding downwardly out of the handles 94.

Optionally, the levers 88 can be lower levers 88 that extend obliquely upward from (e.g., from proximate) the tool surface 82 and/or catch parts 84 to upper levers 89. The upper levers 89 can be joined to one another at their upper ends. The upper connection between the upper levers 89 can be a pivotable junction (e.g., flexible joint, living hinge, area of reduced thickness, or the like) between the upper levers 89. Alternatively, the upper levers 89 may be omitted.

The applicator tool 80 can be an injection-molded, unitary (e.g., single-piece) article formed from an injection-moldable, generally elastic material such as, but not limited to, polycarbonate, polyethylene, or any other suitable injection-moldable material. Alternatively, the applicator tool 80 can be made of metal, metal alloys, steel, or any other suitable materials that can allow for re-sterilization. For example, hinges (e.g., at the junctions between respective portions of the levers 88, 89, and/or links 86) or other suitable connections that allow for relative movements between subparts can be included in the applicator tools 80, such as when the applicator tools are made of relatively rigid materials. As additional examples, a variety of different linkages, levers, and handles of the applicator tool 80 are within the scope of this disclosure.

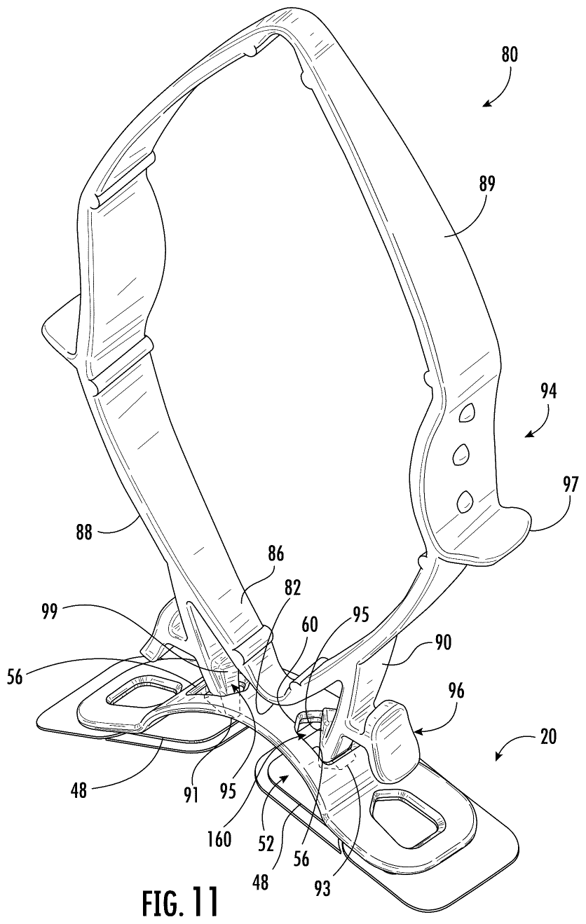

In accordance with the example depicted in FIG. 11, the tissue bridge 20 and applicator tool 80 are cooperatively configured so that the applicator tool can be releasably engaged to (e.g., releasably connected to) the tissue bridge, and the applicator tool can be used to manipulate the tissue bridge as part of a method of mounting the tissue bridge to tissue (e.g., a patient's skin). For example, FIG. 11 depicts the tool surface 82 in opposing-face-to-face relation with (e.g., opposing-face-to-face contact with) an upper surface of the tissue bridge arch 28, and the shanks 90 extending through the tissue bridge inner holes 56. In then configuration depicted in FIG. 11, the engagement shoulders 93 of the outer protrusions or latches 92 are mated against (e.g., positioned against, positioned proximate to, engaged with, in opposing face-to-face contact with, etc.) respective tissue bridge ribs 57 (FIGS. 2-4) with both the tool and tissue bridge in a generally at-rest (e.g., non-deformed, non-extended, etc.) configuration. More generally, the respective catch parts of the applicator tool 80 and tissue bridge 20 are respectively engaged with one another in FIG. 11. It is believed that the tissue bridge ribs 57 may be omitted in which case the engagement shoulders 93 of the outer protrusions or latches 92 can be mated against other suitable surfaces of the tissue bridge 20.

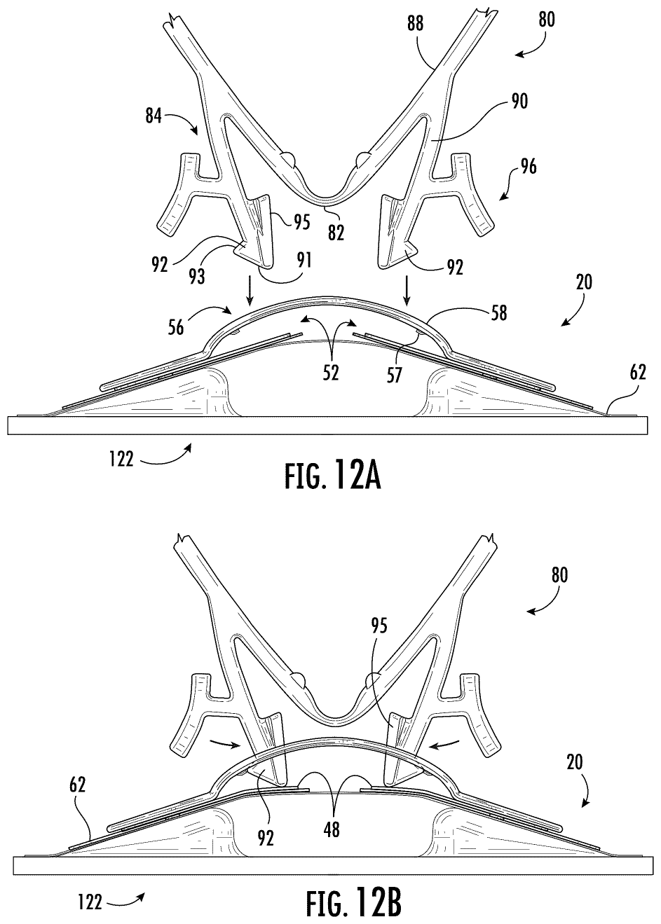

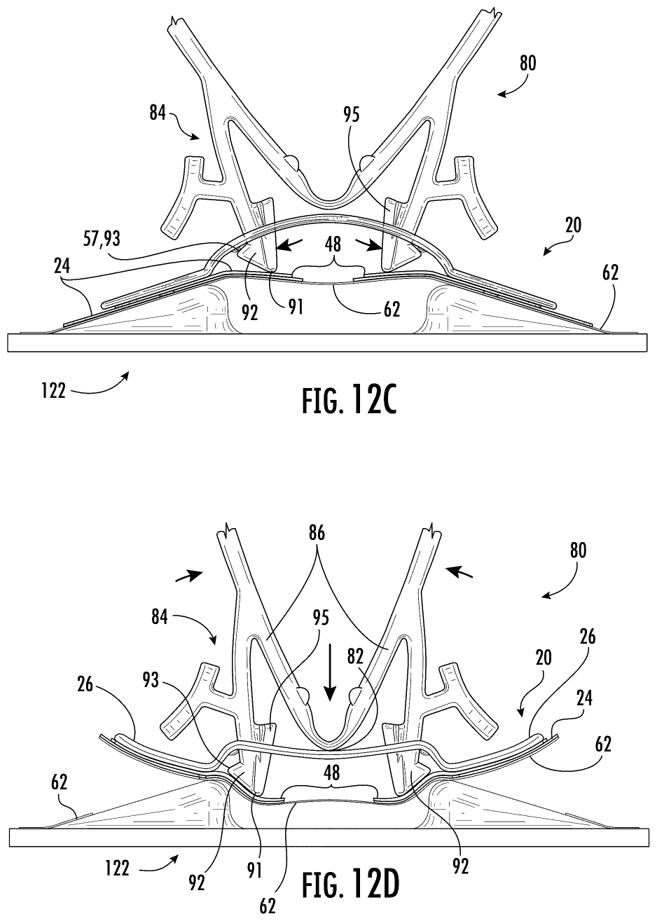

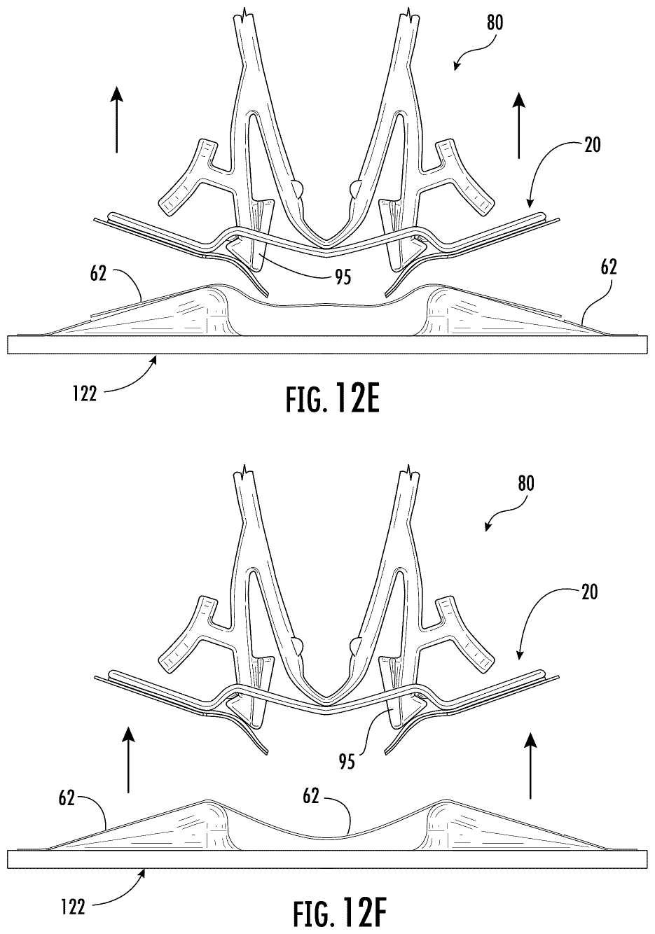

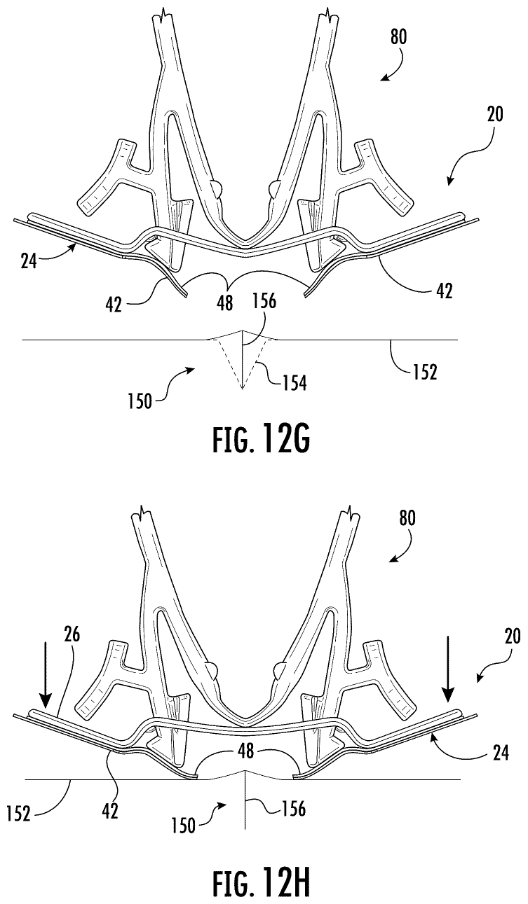

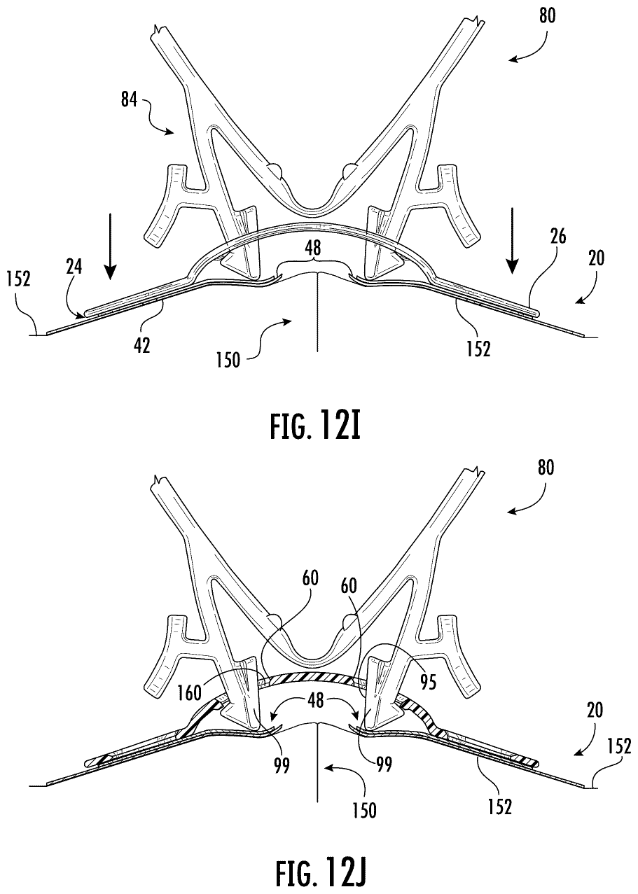

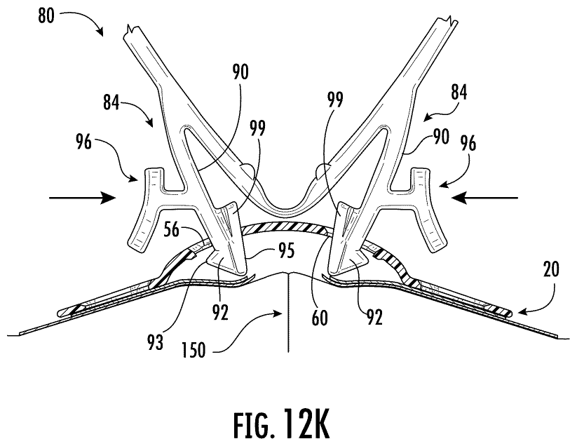

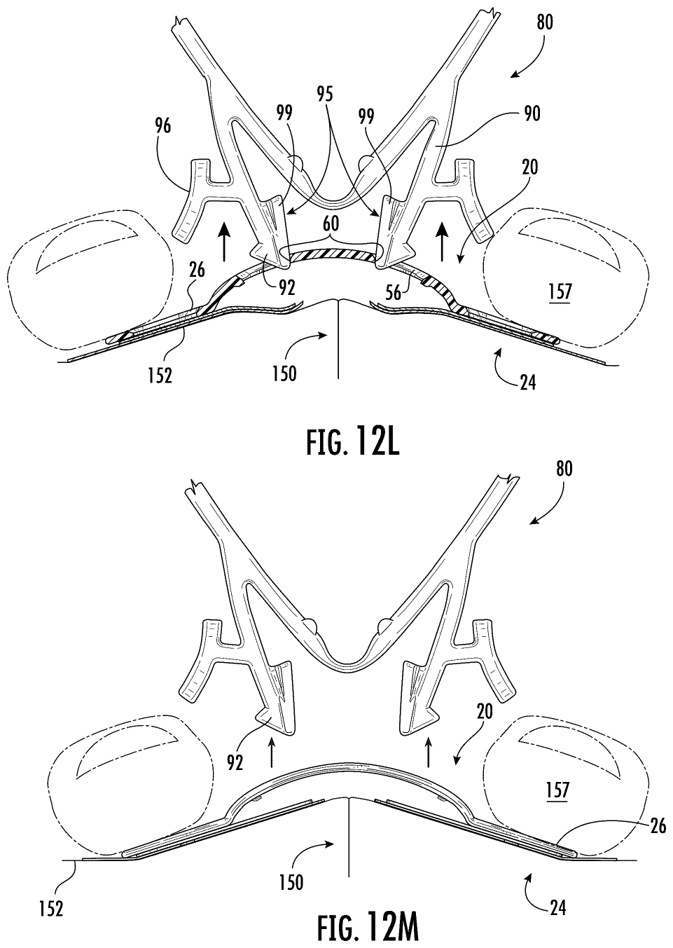

Referring to FIGS. 12A-12F, an exemplary method of using the applicator tool 80 to releasably engage with and remove a tissue bridge 20 from a tray 122 is described. The tray 122 of FIGS. 12A-12F, although not explicitly shown in FIG. 11, may be configured in various ways; for example, housing a plurality of tissue bridges (e.g., a series of tissue bridges arranged in rows, columns, etc.), applicator tools, and/or related tools or devices.

Referring to FIG. 12A, initially, the applicator tool 80 (e.g., in its undeformed, non-extended, or at-rest configuration) can be engaged against or with the tissue bridge 20 (e.g., also in its undeformed or at-rest configuration) by way of relative movement (e.g., schematically depicted by downward pointing arrows) causing increased closeness between the applicator tool 80 and the tray 122 (e.g., movement of the applicator tool toward the tissue bridge mounted on a release liner 62 in the tray). The applicator tool 80 or components thereof (e.g., parts, portions, etc.) may be generally or at least partially elastic and, thus, biased toward its undeformed or at-rest configuration.

With reference to the steps depicted in FIG. 12A and then onto FIG. 12B, in response to the relative movement, at least lower portions of the outer protrusions or latches 92 can enter the tissue bridge receptacles 52 by way of the tissue bridge inner holes 56. That is, lower portions of the latches 92 can enter the receptacles 52 by traveling into and through the tissue bridge inner holes 56. For example, the applicator tool 80 can be pushed (e.g., moved, lowered, etc.) downwardly to releasably engage the tissue bridge 20 in a manner so that the tool latches 92 enter the receptacles 52 by way of the tissue bridge inner holes 56, and optionally also the tool surface 82 engages, or at least becomes more proximate to, the central apex or any other suitable surface of the arch 28. In some embodiments, the tool surface 82 may not (e.g., may never) touch or engage the tissue bridge 20 (e.g., only catch parts 84 directly contact and interact with the tissue bridge).

Also referring to FIGS. 12A and mostly 12B, in response to the relative movement causing increased closeness, the inner protrusions or deflection restrictors 95 of the tool 80 can be positioned in an opposing, face-to-face, relationship with respect to the inner edges 60 (FIGS. 1 and 2) of the tissue bridge holes 56 (FIGS. 1 and 2). As discussed further below, the deflection restrictors 95 can be configured (e.g., arranged, designed, shaped, etc.) to slidably-engage against the tissue bridge inner edges 60 of the tissue bridge holes 56 when moved inwardly to facilitate removal of the tool (e.g., slidable engagement that does not impinge or restrict removal of the tool as discussed in more detail below).

At least a portion of the applicator tool 80 can be deformed during the step of the latches 92 and deflection restrictors 95 entering and/or engaging with the tissue bridge receptacles 52 by way of the tissue bridge inner holes 56 (e.g., releasable engagement). For example, in at-rest states, the distance between the respective engagement shoulders 93 of the latches 92 can be greater than the distance between the hole outer edges 58 (see, e.g., FIG. 2) such that during the connecting of the tool 80 to the tissue bridge 20, initially the lower ends of the shanks 90 are elastically deflected inwardly so that the latches 92 move inwardly (e.g., schematically depicted via inwardly pointing arrows in FIG. 12B), and thereafter the lower ends of the shanks 90 elastically return to or toward their outward configurations so that the latches 92 move (e.g., “snap”) into their latching positions (e.g., in the receptacles 52, beneath the ribs 57 or other suitable structure) and may be releasably connect to the tissue bridge by way of a mechanically interfering arrangement, interference fit, and/or other suitable interaction. The outward movement of the latches 92 relative to one another associated with the snap is schematically represented by a pair of outwardly pointing arrows in FIG. 12C.

The relative movement causing increased closeness between the applicator tool 80 and the tray 122 may be facilitated by a user manually holding the handles 94 and/or other portions of the applicator tool and moving the applicator tool toward the tissue bridge 20 in the tray 122, or the tissue bridge may be supported by any other suitable surface. The lower portions of the catch parts 84 can engage and/or contact respective surfaces of the medial struts 48 in response to the relative movement causing increased closeness between the applicator tool 80 and the tray 122. As a more specific example that can be understood, for example, with reference to FIGS. 4, 10 and 12C, the latch engagement shoulders or surfaces 93 of the tool catch parts can engage with engagement surfaces or ribs 57 of the tissue bridge 20.

Then, for achieving the configuration shown in and FIG. 12D, simultaneously and/or in series, the relative movement causing increased closeness between the applicator tool 80 and the tray 122 can continue (e.g., schematically depicted via the centrally located, downwardly pointing arrow in FIG. 12D). As depicted in FIG. 12D, the handles 94 may be manually squeezed together (e.g., pushed toward one another as schematically depicted via the inwardly pointing pair of arrows in FIG. 12D) so that the applicator tool 80 reconfigures toward its actuated or deformed configuration and applies deforming forces on the tissue bridge 20. As the applicator tool 80 is, for example, simultaneously pushed with greater force against the tissue bridge 20 and caused to deform farther toward its deformed configuration, the applicator tool applies forces against the tissue bridge 20 so that the tissue bridge is responsively deformed toward its strained, deformed, or extended configuration. For example, the applicator tool 80 can apply downward force and laterally outward forces via the catch parts 84. The applicator tool 80 may also apply downward force via the tool surface 82 to the extent that the surface 82 engages (e.g., touches, interacts with, etc.) the tissue bridge 20. Accordingly, the lower surface 82 of the pivotable junction between the links 86 may be referred to as a bearing or contact surface configured to engage the apex of the tissue bridge's central spanning section 30. Alternatively, the bearing or contact surface 82, if present, can be configured differently, for example by being the lower end of a protrusion or other suitable structure extending downwardly from the pivotable junction, and/or the bearing or contact surface 82 can be another suitable portion or structure of the applicator tool 80.

Referring to FIG. 12C, the vertical distance between the medial struts 48 and the tissue bridge inner holes 56 (see, e.g., FIG. 11) can be about the same as, or smaller than, the distance between the latch engagement shoulders 93 and the tips 91 at the lower end of the tool 80. As a result, the latches 92 (e.g., the outer tips 91) can engage the medial struts 48 and cause them to deflect outwardly/downwardly, for example as shown in FIG. 12C. As another example, the distance between the engagement shoulders 93 and the tips 91 can be in a range of from about one to two times the distance between the medial struts 48 and the tissue bridge inner holes 56 in the relaxed configuration of the tissue bridge 20.

Continuing to refer to FIG. 12D, as the exemplary applicator tool 80 is transitioned from its undeformed or at-rest configuration (depicted, e.g., in FIG. 12C) to its actuated or deformed configuration (depicted, e.g., in FIG. 12E), each side of the applicator tool and the associated tissue bridge 20 can deform substantially symmetrically.

In the transition from the configuration of FIG. 12C to the configuration of FIG. 12D, the tissue bridge flanges 26 and outer portions of the foot pads 24 have moved, or more specifically pivoted, away from the tray 122 outer sections. Flap portions of the release liner 62 can be attached to the outer portions of the foot pads 24 by way of the outer adhesive layer 42 (FIG. 6). Therefore, the flaps can be carried by, and pivot with, the outer portions of the foot pads 24. The flaps pivot outwardly relative to a remainder of the release liner 62 that remains fixedly mounted to the tray 122. That is, the flap portions of the release liner 62 can pivot outwardly relative to (e.g., at least partially delaminate from) a reminder of the release liner 62 and the tray 122 in response to respective movement, reconfiguring, and/or the like of the tissue bridge 20 and applicator tool 80. In an exemplary embodiment, the release liner 62 is a support that supports the tissue bridge 20, and flaps thereof can be referred to as a first section of the support, and the reminder of the release liner 62 and/or the tray 122 can be referred to as a second section of the support, or the like.

As another example, in the transition from the configuration of FIG. 12C to the configuration of FIG. 12D, the latches 92 have pushed (e.g., deflected) the medial struts 48 downwardly toward the recessed central section of the tray 122. That is, in an example, the medial struts 48 can pivot downwardly relative to the tissue bridge flanges 26 and a remainder of the foot pads 24 in response to respective movement, reconfiguring, and/or the like of the tissue bridge 20 and applicator tool 80.

Referring to FIG. 12C-12F, the applicator tool 80 and tissue bridge 20 can be cooperatively configured and engaged to one another in a predetermined manner so that, in response to the handles 94 being manually squeezed or pushed closer to one another (e.g., at 12D), at least lower portions of the tool catch parts 84 are moved farther away from one another and the tool surface 82 moves toward a line between the catch parts 84, and this movement of the applicator tool 80 forces the tissue bridge 20 into its fully deformed or extended (e.g., partially flattened) configuration. For example, the manual inward force applied to the opposite sides of the handles 94 to achieve this configuration can be in a range of from more than 0.2 pounds force (0.89 newtons) to less than 2 pounds force (8.9 newtons).

In the transition from the configuration of FIG. 12D to the configuration of FIG. 12E, the release liner 62 typically fully separates from the tissue bridge 20, and the release liner flaps can pivot/fall back into their original positions in response to relative movement causing increased distance between the applicator tool 80 and the tray 122 (e.g., schematically depicted via upward pointing arrows in FIG. 12E). The release liner 62 typically fully separates from the tissue bridge 20 in a manner that fully exposes the outer adhesive layer 42 (e.g., patient contact adhesive identified in FIG. 6), so that there are no remnants of the release liner 62 stuck to the tissue bridge and the outer adhesive layer is ready for being used to secure the tissue bridge to tissue, such as the skin of a patient.

In FIG. 12F, for example, the tissue bridge 20 and applicator tool 80 are engaged to one another, and both the tissue bridge and the applicator tool are in their deformed configurations with the tissue bridge securely grasped or otherwise held by or connected to the applicator tool, so that as the applicator tool is manually moved away from the tray 122 the applicator tool carries the tissue bridge away from the tray (e.g., schematically depicted via upward pointing arrows in FIG. 12F). While the tissue bridge 20 is securely held by (e.g., connected to) the applicator tool 80, the applicator tool can be used to apply the tissue bridge to tissue, such as the skin of a patient, as discussed further below.

Modifications or different arrangements of the steps of using the applicator tool 80 to releasably engage with and remove a tissue bridge 20 from a tray 122 or other suitable surface, as shown in FIGS. 12A-12F, are within the scope of this disclosure. For example, the step of manually squeezing the handles of applicator tool 80 (e.g., illustrated via the pair of generally inward pointing arrows in FIG. 12D) could be performed after the tissue bridge has been separated from release liner 62 and removed from the tray 122 using the applicator tool (e.g., after the step depicted at FIG. 12F). In this regard, the applicator tool 80 could be releasably engaged with the tissue bridge (e.g., as depicted in FIG. 12C) and remain in its undeformed or at-rest configuration until after the applicator tool has been used to carry the tissue bridge away from the tray. For instance, rather than manually squeezing or pushing the handles of the applicator tool closer to one another to apply deforming forces to the tissue bridge as illustrated in step FIG. 12D, the user could tilt and lift the applicator tool 80 such that the tissue bridge 20 separates from the release liner 62 and the applicator tool carries the tissue bridge away from the tray (e.g., similar to the steps illustrated in FIGS. 12D-12F, but with both the tissue bridge and the applicator tool remaining in their at-rest or non-deformed configurations during these steps). In this case, the squeezing of the applicator tool 80 handles could be performed by the user after the removal of the tissue bridge 20 from the tray 122 and release liner 62, and before the tissue bridge is applied to a patient's skin as described in greater detail below.

Referring to FIGS. 12G-12M, a method of using the applicator tool 80 to apply a tissue bridge 20 to tissue 152 on either side of a wound or cut 150 in a patient's skin 152 is described as follows, in accordance with an exemplary embodiment. FIG. 12G schematically depicts with dashed lines 154 the originally spaced apart edges of wound or cut 150, and solid line 156 schematically depicts that the edges of the wound or cut may be manually pushed together prior to applying the tissue bridge 20. The applicator tool 80 holding (e.g., connected to) the tissue bridge 20, both in their respective deformed configurations, can be moved toward the cut 150 so that the tissue bridge 20 extends crosswise to, or more specifically substantially perpendicular to, the length of the cut 150, and the first contact between the tissue bridge and the tissue or skin 152 occurs at the inner end sections or portions of the medial struts 48 on either side of the cut (see, e.g., FIG. 12H).

As shown in FIG. 12H, the applicator tool 80 can continue to be moved or pushed closer to the wound or cut 150 (e.g., illustrated via a pair of downward pointing arrows in FIG. 12H) so that the inner portions of medial struts 48 begin to become adhered to the skin 152 by the outer adhesive layer 42 (e.g., patient contact adhesive), and the tissue bridge flanges 26 and outer portions of the foot pads 24 begin to move or pivot downwardly toward the tissue 152 at opposite sides of the cut 150.

As shown in FIG. 12I, the transmission of force from the applicator tool 80, by way of the catch parts 84, against the medial struts 48 causes the pressure-sensitive adhesive layer 42 of the medial struts 48 to engage against the tissue 152 with sufficient force to cause the medial struts 48 to become adhered to the tissue 152 at opposite sides of the cut 150. Then, as at least partially depicted in FIG. 12I, the manual force on the handles 94 of the applicator tool 80 (e.g., squeezing force) can be reduced by the user so that the tissue bridge 20 begins to return toward its at-rest configuration, and the medial struts 48 become closer together and push the portions of the tissue 152 to which they are adhered toward one another. Then, in response to the tissue bridge 20 returning farther toward its at-rest configuration, the reconfiguring of the tissue bridge causes the flanges 26 to move or pivot so that the outer portions of the foot pads 24 move or pivot downwardly into contact with the tissue 152 at opposite sides of the cut 150 (e.g., illustrated via downward pointing arrows in FIG. 12I). In one example, this contact between the outer portions of the foot pads 24 and the tissue 152 at opposite sides of the cut 150 may occur with sufficient force to cause the pressure-sensitive adhesive layer 42 to securely adhere the outer portions of the foot pads 24 to the tissue 152 at opposite sides of the cut 150.

Thus, in accordance with an exemplary embodiment beginning at FIG. 12H, the medial struts 48 are the first portions of the tissue bridge that contact and are adhesively mounted to the tissue 152 by the user, and this occurs while the tissue bridge 20 is being maintain in its deformed or extended configuration by the user. After the medial struts 48 are adhesively mounted to the tissue 152, the user can relax the squeezing force on the tool handles thereby allowing the tissue bridge 20 to return toward its at-rest configuration and causing the foot pads 24 to become adhesively mounted to the tissue as shown in FIG. 12I. More specifically, when the tissue bridge 20 first contacts or engages against the tissue 152, the point of first contact and adhesive mounting to the tissue can be at the inner end sections or portions of the medial struts 48, and the adhesive mounting of the medial struts 48 to the tissue can occur based upon the medial struts being pushed downwardly by way of the applicator tool 80 as the squeezing force is also being maintained (see, e.g., FIG. 12H). As the deforming forces being applied on the tissue bridge 20 by the applicator tool 80 are reduced, the medial struts 48 move or rotate inwards, thus centrally pulling and/or pushing the tissue portions 152 to which they are adhesively mounted, and this action by the medial struts 48 occurs before the remainder or outer portions of the foot pads 24 are adhesively attached to the tissue. At this intermediate point in which the medial struts 48 are at least partially attached to the tissue 152 and have moved inwards, and the remainder or outer portions of the foot pads 24 are not yet attached to the tissue, the shear stress and/or strain on predetermined tissue (i.e., tissue that is lateral to the lateral-most contact point between the medial strut and the tissue) is distributed laterally and in a gradual manner. Then, when the remainder of the foot pads 24 are adhered to the tissue 152, the predetermined tissue underneath and at the lateral edges or outer edges of the foot pads 24 is secured (e.g., adhered to the foot pads) in a state in which the stress and/or strain in the predetermined tissue is distributed laterally and in a gradual manner, which seeks to prevent sudden, high sheer stress at the lateral edges (e.g., opposite ends) of the tissue bridge 20.

FIG. 12J is like FIG. 12I except that in FIG. 12J the tissue bridge 20 is depicted in cross-section, with the cross-section taken along line 4-4 of FIG. 2, or the like. When the tool 80 and tissue bridge 20 are cooperatively configured as depicted in FIG. 12J and FIG. 11, there are gaps or spaces 160 defined between the inner protrusions or deflection restrictors 95 of the tool 80 and the inner edges 60 of the tissue bridge holes 56 (e.g., engagement faces or surfaces 99 of the deflection restrictors 95 are respectively in spaced-apart, opposing face-to-face relation with respect to the inner edges 60 of the tissue bridge holes 56).

FIG. 12K also includes a cross-sectional view of the tissue bridge 20 taken along line 4-4 of FIG. 2, or the like. In the example depicted in FIG. 12K, the shanks 90 or release pads 96 (if present) may be touched or contacted (e.g., squeezed together) by the user causing at least lower portions of the catch parts 84 to move closer to one another, as schematically depicted via inwardly pointing arrows, so that the catch parts (e.g., shoulders 93) disengage from the tissue bridge and can thereafter be passed through and out of the tissue bridge holes 56 (see, e.g., FIGS. 12L-12M). As depicted in FIGS. 12K and 12L, squeezing together the release pads 96 or shanks 90 can cause the backslopes (e.g., the engagement surfaces or faces 99 of the tool deflection restrictors 95) to respectively engage or contact the tissue bridge inner edges 60 such that the gaps or spaces 160 (see, e.g., FIG. 12J) have been at least partially obstructed, closed, or at least substantially closed.

The deflection restrictors 95 can include or take the form of a molded or built-up portion extending inwardly from the shanks 90. The deflection restrictors 95 can be configured to at least partially facilitate removal of (e.g., disconnection of) the tool 80 from the tissue bridge 20; e.g., preventing or decreasing the likelihood of an impingement (e.g., an undesirable mechanically interfering arrangement and/or connection between the tool and the tissue bridge) when one or both of the catch parts 84 are moved (e.g., manually squeezed) to an inner position or configuration as illustrated in FIG. 12K, and the tool is subsequently moved or lifted away from the tissue bridge (e.g., movement causing increased distance between the tool 80 and the tissue bridge 20). As a contrasting example, in some configurations without the deflection restrictors 95, if the catch parts 84 were squeezed too closely together during the process of tool removal, areas of contact between the catch parts 84 and the tissue bridge (e.g., at, beneath, and/or lowerly adjacent the tissue bridge inner edges 60) may restrict relative movement (e.g., may cause non-slidable engagement between the catch parts 84 and tissue bridge edges 60 or adjacent surfaces of the tissue bridge) so that removal of the tool from the tissue bridge would be restricted. As a more specific example of the situation in which the deflection restrictors 95 were omitted and the catch parts 84 were squeezed too closely together during the process of tool removal, the tissue bridge inner edges 60 and/or tissue bridge surface(s) adjacent the tissue bridge inner edges 60 may define an “undercut,” and respective portions of the catch parts 84 extending beneath the undercuts may engage the central portion of the tissue bridge from below so that removal of the tool from the tissue bridge would be restricted.

In contrast, in the exemplary embodiment shown in FIG. 12K, respective surfaces of the deflection restrictors 95 of the applicator tool 80 are shaped to slidably interact with the shape of the hole inner edges 60 at or about the chevron (e.g., inverted V-shape of the holes defined by convergent inner edges 60). Notably, the deflection restrictors 95 being configured to interact with the tissue bridge inner holes 56 (e.g., slidable engagement) can reduce pinch forces (e.g., undesirable connection) and/or provide a shorter path of travel (e.g., shorter overall height or length) for the shanks. In a general sense, the lower portions of the catch parts 84 (in combination with the deflection restrictors 95) may visually resemble an arrow. The upper portions of the catch parts 84 or shanks 90 may be narrower (e.g., having a narrowed waist) as compared to the outer and inner protrusions 92, 95 (e.g., latches and deflection restrictors) so that the upper portions of the shanks can be more readily deformed (e.g., in response to touching (e.g., squeezing together) the release pads 96, shanks 90, or other suitable portions of the tool 80).

At least partially reiterating from above, a method of the deflection restrictors 95 engaging against one or more of the corresponding tissue bridge inner edges 60 is described in the following, in accordance with an embodiment if this disclosure. Referring to FIG. 12K, in response to any suitable inward forces being applied (e.g., by a user to the shanks 90, release pads 96, and/or other suitable portions of the tool 80) so that the distance between the latches 92 has been reduced to or beyond a predetermined magnitude (e.g., so that the latches 92 are no longer connecting the tool 80 to the tissue bridge 20 and the tool can be removed from the tissue bridge), the convex corners (defined by the confluence (e.g., convergence) of respective tool engagement faces 99) respectively mate into (e.g., nest into) and engage the concave corners (defined by the confluence of respective tissue bridge edges 60 (FIGS. 1-2)).

Referring to FIG. 12L, in response to any suitable inward forces continuing to being applied (e.g., by a user to the shanks 90, release pads 96, and/or other suitable portions of the tool 80 as schematically depicted by inwardly pointing arrows in FIG. 12K) during all or part of the removal of the tool 80 from the tissue bridge 20, the convex corners (e.g. respectively defined by tool engagement faces 99) can respectively remain mated into and engaged in the concave corners (respectively defined by tissue bridge edges 60) so that sliding contact or engagement occurs between respective tissue bridge inner edges 60 and tool engagement faces 99. These sliding engagements can extend in respective vertical or predetermined inclined directions or planes configured in a manner that seeks to streamline disengaging and removing the applicator tool 80 from the force modulating tissue bridge 20 (e.g., by inhibiting the tool from being inadvertently reconnected to the tissue bridge as a result of any occurrence of the catch parts 84 becoming too close to one another, by restricting the catch parts 84 from engaging beneath tissue bridge inner edges 60, by providing ejecting forces that seek to push the catch parts outwardly through the tissue bridge holes 56, and/or etc.) For example, the subject sliding contacts, tool engagement faces 99, and convex corners can extend vertically or be inclined to respectively extend convergently toward one another in an upward direction, or the like, as discussed further below.

In an example, for each convex corner, an angle (convex corner angle) is defined between the tool engagement faces 99 that define the convex corner, for each concave corner an angle (concave corner angle) is defined between the tissue bridge edges 60 that define the concave corner, the convex and concave corner angles may be about the same, or the convex corner angle may be smaller than the concave corner angle. One or more of the convex and concave corners can be relatively sharp corner(s) and/or rounded corner(s). As an example, any such rounded corners may be defined by a single curved tissue bridge edge 60 or a single curved tool engagement face 99, such that tissue bridge edges 60 may not be provided in adjacent pairs and/or the engagement faces 99 may not be provided in adjacent pairs. More generally, as compared to the embodiments depicted in the drawings, there can be a greater or lesser number of the tissue bridge edges 60 or tool engagement faces 99.

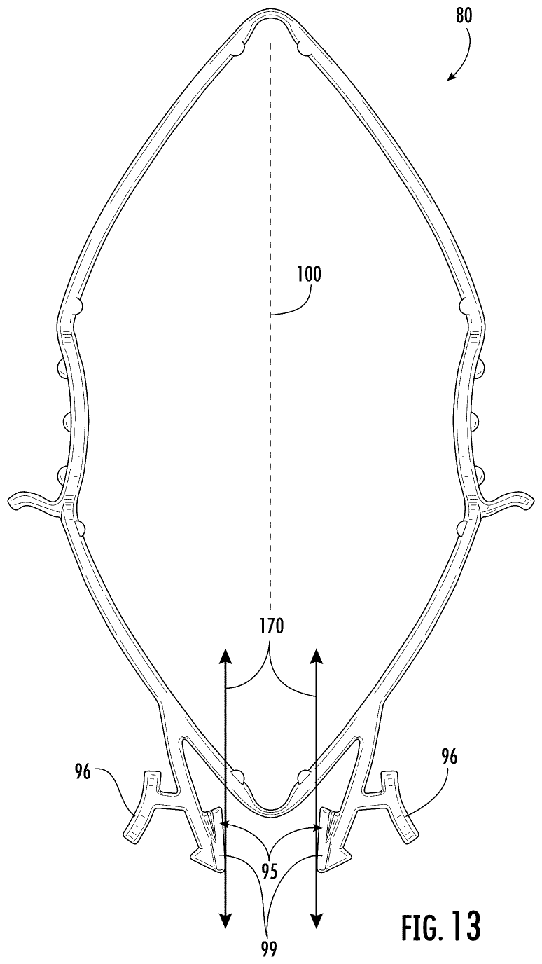

As schematically depicted in FIG. 13, the deflection restrictors 95 may be formed (e.g., molded, shaped, designed, etc.) so that respective inner faces or surfaces (e.g., contact surfaces or more specifically engagement faces 99) are parallel or approximately parallel to each other, a plumb line passing through the applicator/tissue-bridge assembly as schematically illustrated via reference lines 170, and/or an axis 100 of the tool 80. The reference lines 170 are depicted as being parallel to and positioned on opposite sides of the axis 100 of the tool. In, for example, FIG. 13, the axis 100 is schematically represented by a dashed line, and the axis 100 may be a lengthwise axis of the tool 80, an axis between opposite sides of the tool, an axis about which one or more hinges (e.g., upper and lower pivotable junctions or living hinges of the tool) flex, and/or other suitable axes.

The tool 80 can be configured so that, in response to touching (e.g., squeezing together) the release pads 96 or otherwise moving the deflection restrictors 95 of the tool into an inner configuration allowing removal of the tool 80 from the tissue bridge 20, slidable contact between the tissue bridge inner edges 60 and deflection restrictors 95 can be along or about a vertical plane (e.g., parallel to lines 170). Thus, the tool 80 can be lifted upward by the user and one or more of the deflection restrictors 95 and/or portions thereof (e.g., backslopes or engagement faces 90) can slide against corresponding tissue bridge inner edges 60 thereby facilitating (e.g., guiding, preventing impingement, allowing for, etc.) removal.

The deflection restrictors 95 of the applicator tool 80 and/or the corresponding inner edges 60 of the tissue bridge holes 56 can, respectively, be modified or altered to include additional and/or different features, and such features may provide different or additional functionality relating to removal (e.g., suitably touching (e.g., pressing) the release pads 96 as shown in FIG. 12K). For example, moving or extending the inner edges 60 of the tissue bridge holes 56 inwards (e.g., further toward the mid-point or central area of the tissue bridge 20) can allow for different interactions during slidable contact (e.g., in response to touching (e.g., pressing) release pads 96). As another example, as schematically depicted in FIG. 14, deflection restrictors 95 may be formed with non-parallel inner or tool engagement faces 99; e.g., with a backslope or inclined angle in relation to each other and/or vertical lines 170.

The backslopes defined, for example, by the deflection restrictors 95 can be formed with varying slopes (e.g., ramp, incline, rise, gradient, decline, etc.) such that slidable contact with corresponding tissue bridge inner edge portions 60 occurs at or along an angle of slidable contact, for example an angle of about 180 degrees or less. For example, in FIGS. 14 and 15 angles of, or associated with, the subject sliding contact are identified by the symbol Θ (e.g., theta in the Greek alphabet). For example, it is believed the angles Θ may be defined between vertical (e.g., the axis 100 of the tool) and the backslopes or engagement faces 99 and/or the upright edges of the convex corners respectively at the confluence of the engagement faces 99. As examples, it is believed that the angles Θ can be about 180 degrees or less, about 180 degrees or less, in a range of from about 180 degrees to about 90 degrees, from about 180 degrees to about 120 degrees, from about 170 degrees to about 130 degrees, from about 160 degrees to about 140 degrees, about 150 degrees, or any values or subranges therebetween, including both with and without the adjective “about.”

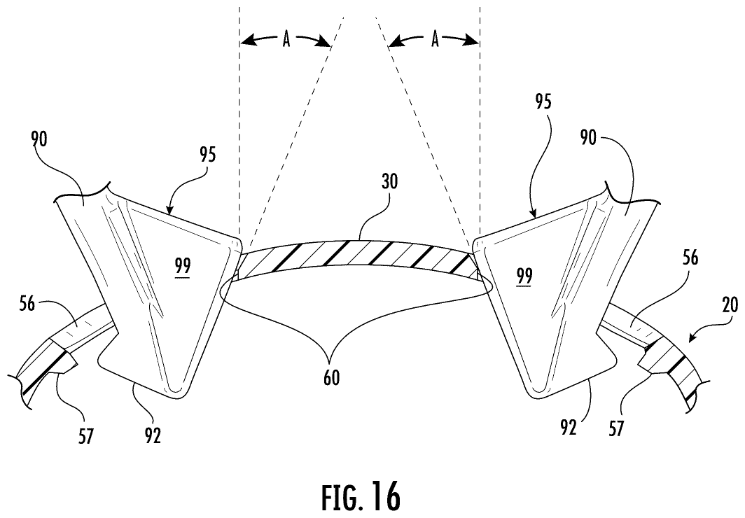

FIG. 15, which is similar to the view and/or step of FIG. 12K, schematically depicts an example of the deflection restrictors 95 slidably contacting tissue bridge inner edges 60 along angles Θ of about 150 degrees in response to release pads 96 (or other suitable parts) being pressed toward one another (schematically illustrated via inwardly pointing arrows). As shown in FIG. 15, as opposed to slidable contact along or about a generally vertical line or plane 170, slidable contact at and/or along an angled slope can result in a ramping effect of various types or degrees such that touching or inwardly pressing the release pads 96 causes the applicator tool 80 to be moved (e.g., ejected) away from the tissue bridge (e.g., lessening or eliminating lifting by the user). Thus, the subject slidable contact can occur along an angled slope to assist in the process or step of removal; e.g., moving and/or ejecting the tool 80 away from the tissue bridge via ramping effect of the slope. In this regard, it is believed the angle A (see, e.g., FIG. 16) of opposing face-to-face-sliding contact between the tool engagement faces 99 (e.g. backslopes) and corresponding portions of the tissue bridge inner edges 60 can, for example, be greater than 0 degrees and less than 90 degrees, from about 30 degrees to about 60 degrees, from about 40 degrees to about 50 degrees, about 45 degrees, or any values or subranges therebetween, including both with and without the adjective “about,” such that the applicator tool 80 moves away and/or ejects from (e.g., is pushed away from) the tissue bridge 20 in response to the user touching or inwardly pressing the release pads 96. In such scenarios, it is believed that the applicator tool 80 is forced or pushed outwardly in a direction away from the tissue bridge 20 by normal forces applied by the tissue bridge inner edges 60 against the tool engagement faces 99.

As another example of various angles defined by the tool 80, as schematically depicted in FIG. 14, for each tool catch part 84 and its associated deflection restrictor 95, it is believed that the angle numeral 8 can be at least about 15 degrees, about 44 degrees, less than 90 degrees, in a range of from about 10 degrees to about 70 degrees, from about 20 degrees to about 60 degrees, from about 30 degrees to about 50 degrees, from about 35 degrees to about 88 degrees, or any values or subranges therebetween, including both with and without the adjective “about.”

Referring to FIGS. 12L to 12M, the applicator tool 80 can be removed from the tissue bridge 20 so that the tissue bridge remains mounted over the wound (e.g., cut, incision, etc.) or scar 150. The applicator tool may then, for example, be used to install another tissue bridge. In addition, a user can push down manually with their fingers 157 on the tissue bridge flanges 26, for example, with sufficient force to ensure that the pressure-sensitive adhesive layer 42 securely adheres the foot pads 24 to the tissue 152 at opposite sides of the cut 150. The tissue bridge 20 can be mounted to the tissue 152 in a manner such that the tissue bridge and tissue apply force against one another, and the force applied by the tissue typically restricts the tissue bridge from fully returning to its at-rest configuration. As a result, the tissue bridge 20 applies forces to the tissue 152 by way of the foot pads 24 in a manner that can, for example, reduce tension in the tissue, help close the wound 150, help inhibit wound reopening, and/or inhibit scar disfiguring (e.g., widening). In the example shown in FIG. 12M, the tissue 152 proximate the scar and/or wound 150 bulges into the central area over which the arch 28 extends.

The tissue bridge 20, release liner 62, and applicator tool 80 can be configured differently than discussed above. As examples, one or more of the layers of the tissue bridge 20 can be omitted or configured differently than discussed above, and/or one or more features of the tool 80 (e.g., a deflection restrictor 95) may be omitted or configured differently than discussed above.

Modifications or rearrangements of the steps described above and shown in FIGS. 12K-12M are within the scope of this disclosure. For example, without inwardly pressing pads 96 as schematically depicted in FIG. 12K via inwardly pointing arrows, the user can instead press down on the foot pads 24 with their fingers 157 (e.g., FIG. 12L) with sufficient force whereby the tissue bridge 20 further deforms causing the tool deflection restrictors 95 to engage or contact the inner edges 60 of the tissue bridge holes 56 such that the gaps or spaces 160 have been closed. In this instance, the pressing and further deformation by the user corresponds with the user lightly engaging or suitably touching the pads 96 to thereby accomplish removal of the tool (e.g., FIGS. 12L-12M). Alternatively, other portions of the applicator tool (i.e., components other than pads 96) may be engaged simultaneously with the pressing and further deformation by the user to accomplish removal.

In another alternative to simultaneous application of forces or squeezing the pads 96 toward one another in the manner schematically depicted by the inwardly pointing arrows of FIG. 12K, the user may tilt (e.g., gently tilt or pivot) the tool 80 toward either end of the tissue bridge 20 (i.e., one end or the other) while touching (e.g., pressing) one or more of the release tabs or pads 96 to accomplish removal of the tool. For example, in an example of a method, the tool 80 can be detached from the tissue bridge 20 by rocking (e.g., pivoting as a whole) the applicator (relative to the tissue bridge) toward one side or end of the tissue bridge while pressing only one of release tabs or pads 96 (e.g., rocking or pivoting while not simultaneously forcibly touching both of the release pads 96). When using this method, typically one tool catch part 84 is removed from the tissue bridge 20 at a time; the tool catch parts are not simultaneously removed from the tissue bridge; the tool catch parts are serially (i.e., one after the other) removed from the tissue bridge. In accordance with this method, a first of the catch pins or parts 84 can remain latched to and/or engaged against the tissue bridge 20 while the second of the catch pins or parts is pressed inwardly and pivoted (with the tool) upwardly and away from (out of) the respective tissue bridge hole 56. After the second catch part 84 is released (e.g., is out of or substantially free from the respective tissue bridge hole 56), the tool 80 as a whole can be moved away from (e.g., it is believed the tool as a whole may be translated away from) the tissue bridge 20, so that the second catch part 84 is moved upwardly and away from (out of) the respective tissue bridge hole 56. Accordingly, one of the backslopes or deflection restrictors 95 may not be utilized or may be omitted, as discussed further below.

FIGS. 7-13 can be described as depicting a tool 80 of a first embodiment of this disclosure, FIGS. 11 and 12A-12B can be described as depicting aspects of a method of the first embodiment, FIG. 14 can be described as depicting a tool of a second embodiment of this disclosure, and FIG. 15 can be described as depicting aspects of a method of the second embodiment. The first and second embodiments can be alike, except for variations noted and variations that will be apparent to those of ordinary skill in the art.

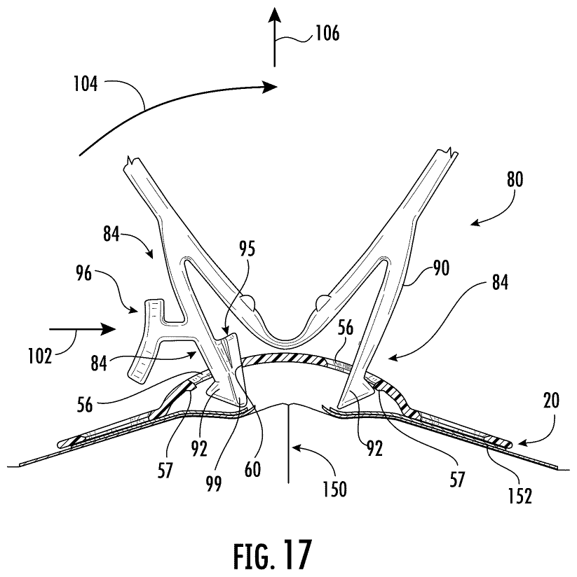

FIG. 17 depicts an example of a lower portion of a tool 80, and further schematically depicts aspects of a method of removing the tool 80 from a tissue bridge 20 in a manner as at least partially alluded to above and in accordance with a third embodiment of this disclosure. The first, second, and third embodiments can be alike, except for variations noted and variations that will be apparent to those of ordinary skill in the art.

The third embodiment of the tool 80, a portion of which is depicted in FIG. 17, includes only one deflection restrictor 95 (e.g., deflection-restricting backslope) and, thus, the tool is asymmetrical. An example of a method of removing the tool 80 from the tissue bridge 20 is described in the following with reference to FIG. 17 and in accordance with the third embodiment. The catch pin or part 84 without a deflection restrictor 95 (e.g., the catch part to the right in FIG. 17) can (i) remain latched to and/or engaged against the tissue bridge 20 while the catch part with or associated with the deflection restrictor 95 (e.g., the catch part to the left in FIG. 17) is pressed inwardly (schematically represented by arrow 102 in FIG. 17) so that the deflection restrictor engages the respective tissue bridge inner edge(s) 60 and, thus, is unlatched, and (ii) then the tool as a whole is pivoted so that the catch part with the deflection restrictor is pivoted upwardly and away from (out of) the respective tissue bridge hole 56. This pivoting is schematically represented in FIG. 17 by arrow 104. At the appropriate time, the pivoting schematically represented by arrow 104 can be achieved, for example, by a user manually touching and applying force to one or more suitable portions (e.g., the left release pads 96) of the tool 80. During the pivoting schematically represented by the arrow 104, the tissue bridge rib 57 engaged against the catch part 84 without the deflection restrictor 95 functions as, and/or other suitable portion(s) of the tissue bridge 20 can function as, the fulcrum about which the tool 80 pivots.

Responsive to the motions schematically represented by arrows 102 and 104 in FIG. 17 (and typically accompanied by sliding engagement between the respective tissue bridge inner edge(s) 60 and the tool backslope (e.g., engagement face(s) 99)), the catch part 84 with the deflection restrictor 95 is released from and moved at least partially away from the tissue bridge 20 (e.g., becomes positioned outside of and/or is substantially free from the respective tissue bridge hole 56). After the catch part 84 with the backslope of deflection restrictor 95 is released moved relative to the tissue bridge 20 as discussed immediately above, the tool 80 can be further removed from the tissue bridge 20 by moving the tool 80 as a whole upwardly and/or upwardly in an inclined direction (e.g., as schematically represented by arrow 106 in FIG. 17), so that the catch part 84 without the deflection restrictor 95 is moved upwardly and away from (out of) the respective tissue bridge hole 56.

It is believed that the above-described method of removing the third embodiment tool 80 from the tissue bridge 20 can optionally also be performed with at least some of the above-discussed versions of the tools 80 of the first and second embodiments that each include two of the deflection-restricting features (e.g., backslopes, deflection restrictors 95, or the like), wherein only one of the deflection-restricting features is utilized during the removal of the tool.

To supplement the present disclosure, this application incorporates by reference the entire disclosure of each of the following patent application publications: United States Patent Application Publication No. 2014/0128819, United States Patent Application Publication No. 2014/0227483, and United States Patent Application Publication No. 2019/0133582. This application also incorporates by reference the entire disclosure of U.S. Provisional Patent Application No. 63/274,132 filed Nov. 1, 2021.

It is within the scope of this disclosure for one or more of the terms “substantially,” “about,” “approximately,” and/or the like, to qualify each adjective and adverb of the foregoing disclosure, to provide a broad disclosure. As an example, it is believed those of ordinary skill in the art will readily understand that, in different implementations of the features of this disclosure, reasonably different engineering tolerances, precision, and/or accuracy may be applicable and suitable for obtaining the desired result. Accordingly, it is believed those of ordinary skill will readily understand usage herein of the terms such as “substantially,” “about,” “approximately,” and the like.

In the specification and/or figures, embodiments of the invention have been disclosed. The present invention is not, however, limited to such exemplary embodiments. For example, the present invention is not limited to the specific details (e.g., dimensions and ratios) disclosed. The use of the term “and/or” includes any and all combinations of one or more of the associated listed items. The figures may be schematic representations and are not necessarily drawn to scale. Unless otherwise noted, specific terms have been used in a generic and descriptive sense and not for purposes of limitation.

1. A tool configured to interact with a medical article, the tool comprising:
first and second levers pivotably connected to one another;
first and second catch parts configured to at least partially releasably connect the tool to a medical article, wherein:
the first and second catch parts respectively comprise first and second shanks respectively connected to the first and second levers, and first and second outer protrusions respectively extending outwardly from the first and second shanks,
the tool is configured to be connected to the medical article at least partially in response to an increased distance between the first and second outer protrusions, and
the tool is configured to be disconnected from the medical article at least partially in response to a reduced distance between the first and second outer protrusions; and
a first inner protrusion extending inwardly from the first shank, wherein the first inner protrusion is configured to engage at least one portion of the medical article to restrict how close the first and second catch parts can be to one another while the tool is being removed from the medical article.

2. The tool according to claim 1, further comprising a second inner protrusion extending inwardly from the second shank, wherein the first and second inner protrusions are configured to slidingly engage respective portions of the medical article to restrict the tool from inadvertently being in a mechanically interfering arrangement with the medical article by way of any occurrence of the first and second catch parts becoming too close to one another.

3. The tool according to claim 1, further comprising a second inner protrusion extending inwardly from the second shank, wherein the first and second inner protrusions are configured to slidingly engage the respective portions of the medical article to restrict the catch parts from engaging beneath respective portions of the medical article while the tool is being removed from the medical article.

4. The tool according to claim 1, further comprising a second inner protrusion extending inwardly from the second shank, wherein:
engagement surfaces respectively of the first and second inner protrusions are configured to slidingly engage respective portions of the medical article to restrict how close the first and second catch parts can be to one another while the tool is being removed from the medical article; and
the engagement surfaces:
extend vertically, or
are inclined and extend convergently toward one another in an upward direction.

5. The tool according to claim 1, further comprising a second inner protrusion extending inwardly from the second shank, wherein:
the first and second shanks respectively extend downwardly from the first and second levers, and
the first and second inner protrusions extend farther upwardly than the first and second outer protrusions.

6. The tool according to claim 1, comprising a pivotable junction, and further comprising a second inner protrusion extending inwardly from the second shank, wherein:
the first and second levers respectively extend upwardly and outwardly from the pivotable junction, and
the first and second inner protrusions extend at least as high as a portion of the pivotable junction.

7. The tool according to claim 1, wherein:
the first inner protrusion comprises an upright corner and upright engagement surfaces extending away from the upright corner; and
the engagement surfaces extend divergently from one another in a direction away from the corner.

8. The tool according to claim 1, wherein:
the first inner protrusion comprises a shoulder extending inwardly from the first shank, and
an engagement surface of the first inner protrusion extends downwardly from the shoulder.

9. The tool according to claim 1, further comprising a second inner protrusion extending inwardly from the second shank, wherein the first and second inner protrusions are respectively configured to contact the medical article at an angle between about thirty degrees and about sixty degrees, wherein the angle is defined between the inner protrusion and the medical article.

10. The tool according to claim 1, further comprising a second inner protrusion extending inwardly from the second shank, wherein the first and second inner protrusions are respectively configured to slidably contact the medical article at an angle between about zero degrees and about ninety degrees, wherein the angle is defined between the inner protrusions and the medical article.

11. The tool according to claim 10, wherein the tool is configured to eject from the medical article in response to the first and second inner protrusions being moved inwardly to slidably contact the medical article.

12. A system comprising the tool according to claim 1 in combination with the medical article, wherein:
the medical article comprises a central portion and spaced apart holes extending through the central portion;
the inner edges respectively are inner edges of the holes;
the shanks are configured to respectively extend at least partially into the holes; and
the tool is configured to release from the medical article in response to at least the first and second shanks being moved inwardly toward one another to an inner configuration in which the first inner protrusion and a second inner protrusions of the tool are in opposing face-to-face contact respectively with the inner edges of the holes.

13. A tool configured to interact with a medical article, the tool comprising:
first and second levers pivotably connected to one another; and
first and second catch parts, comprising:
first and second shanks respectively extending downwardly from the first and second levers, and
first and second outer protrusions respectively extending outwardly from the first and second shanks and configured to releasably engage beneath first respective portions of a medical article when the tool is releasably connected to the medical article; and
a first inner protrusion extending inwardly from the first shanks, wherein the first inner protrusion comprises an engagement surface configured to engage at least one portion of the medical article while the tool is being removed from the medical article, and the engagement surface extends vertically or extends outwardly in a downward direction.

14. The tool according to claim 13, wherein:
the first inner protrusion comprises a shoulder extending inwardly from the first shank, and
the engagement surface of the first inner protrusion extends downwardly from the shoulder.

15. The tool according to claim 13, wherein:
the tool is configured to interact with the medical article in response to the first and second levers being moved toward one another; and
the tool is configured to release from engagement with the medical article in response to at least the first shank being moved inwardly such that the first inner protrusion contacts the medical article.

16. The tool according to claim 13, further comprising a second inner protrusion extending inwardly from the second shank, wherein the first and second inner protrusions are configured to slidably contact the medical article as the tool is moved away from the medical article.

17. The tool according to claim 13, further comprising a second inner protrusion extending inwardly from the second shank, wherein the first and second inner protrusions are configured to resist interlocking engagement with the medical article as the tool is moved away from the medical article.

18. The tool according to claim 13, further comprising a second inner protrusion extending inwardly from the second shank, wherein the first and second inner protrusions are respectively configured to slidably contact the medical article about an approximately vertical plane.

19. The tool according to claim 13, further comprising a second inner protrusion extending inwardly from the second shank, wherein the first and second inner protrusions are respectively configured to contact the medical article at an angle between a range of zero and ninety degrees, wherein the angle is defined between the inner protrusions and the medical article.

20. The tool according to claim 13, further comprising a second inner protrusion extending inwardly from the second shank, wherein the first and second inner protrusions are respectively configured to contact the medical article at an angle between thirty and sixty degrees, wherein the angle is defined between the inner protrusions and the medical article.

21. A system comprising the tool according to claim 13 in combination with a medical article, wherein:
the medical article comprises a central portion and spaced apart holes;
the shanks are configured to respectively extend at least partially into the holes; and
the tool is configured to release from the medical article in response to at least the first and second shanks being moved inwardly toward one another to an inner configuration in which the first inner protrusion and a second inner protrusions of the tool are in opposing face-to-face contact respectively with the inner edges of the holes.

22. A system comprising:
a medical article comprising a central portion and spaced apart holes in the central portion; and
a tool configured to releasably engage and interact with a medical article, wherein the tool comprises:
first and second levers pivotably connected to one another,
first and second shanks respectively connected to the first and second levers and configured to respectively extend at least partially into the holes,
first and second outer protrusions respectively extending outwardly from the first and second shanks,
first and second inner protrusions respectively extending inwardly from the first and second shanks, and
a contact surface positioned between the first and second shanks and configured to engage an upper surface of the central portion that is positioned between the holes, wherein the first and second inner protrusions:
extend to at least as high as the contact surface,
extend farther upwardly than the first and second outer protrusions, and
are configured to respectively be in sliding, opposing face-to-face contact with inner edges of the holes while the tool is being released from engagement with the tissue bridge.

23. The system according to claim 22, wherein:
the medical article comprises a tissue bridge configured to reduce tension in tissue of a patient;
the first inner protrusion comprises a shoulder extending inwardly from the first shank;
the first inner protrusion comprises an engagement surface extending downwardly from the shoulder and configured to be in sliding, opposing face-to-face contact with an inner edge of a first hole of the holes while the tool is being released from engagement with the tissue bridge;
the second inner protrusion comprises a shoulder extending inwardly from the second shank; and
the second inner protrusion comprises an engagement surface extending downwardly from the shoulder of the second inner protrusion and configured to be in sliding, opposing face-to-face contact with an inner edge of a second hole of the holes while the tool is being released from engagement with the tissue bridge.
